(19) United States
(12) Patent Application Publication
(10) Pub. No.: US 2024/0360147 A1
(43) Pub. Date: Oct. 31, 2024

(54) Process for Synthesizing Naphthyridine Derivatives and Intermediates Thereof

(71) Applicant: AMGEN INC., Thousand Oaks, CA (US)

(72) Inventors: Athimoolam Arunachalampillai, Tamilnadu (IN); Richard David Crockett, Thousand Oaks, CA (US); Robert P. Farrell, Thousand Oaks, CA (US); Ted Charles Judd, Granada Hills, CA (US); Adrian Ortiz, Oak Park, CA (US); Joanna Robinson, Los Angeles, CA (US); Carolyn S. Wei, Oak Park, CA (US); Kumiko Yamamoto, Thousand Oaks, CA (US); Diana Catherine Fager, Lexington, MA (US); Heather Claire Johnson, Jamaica Plain, MA (US); Neil Fred Langille, Sudbury, MA (US); Liang Zhang, Sharon, MA (US)

(21) Appl. No.: 18/687,347

(22) PCT Filed: Aug. 30, 2022
(86) PCT No.: PCT/US22/75648

§ 371 Date: Feb. 28, 2024

Related U.S. Application Data
(60) Provisional application No. 63/354,464, filed on Jun. 22, 2022.
(60) Provisional application No. 63/238,337, filed on Aug. 30, 2021.

(51) Int. Cl. C07D 491/147

(52) U.S. Cl. CPC C07D 491/147

(57) ABSTRACT

The disclosure provides processes for preparing Compound A, Compound E, Compound I, salts thereof, and/or stereoisomers thereof, as described herein.

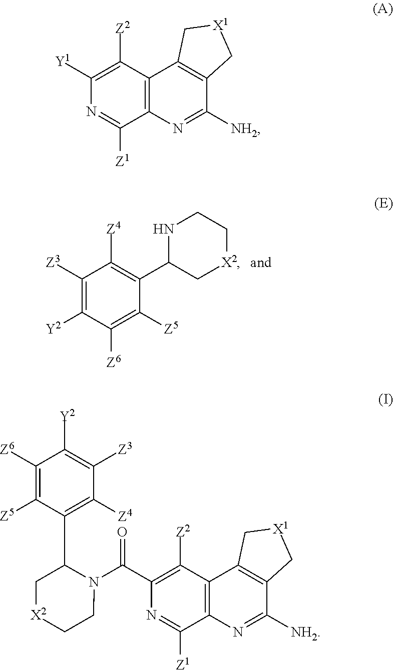

### BACKGROUND

Naphthyridine derivatives and intermediates have been shown to be important in a number of biological applications. In order to investigate their efficacy, large quantities of the materials are needed. As such, there is a need for efficient, cost-effective processes for preparing naphthyridine derivatives that are amenable to large scale.

### SUMMARY

The disclosure provides processes for preparing Compound A, or a salt thereof,

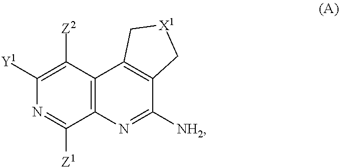

wherein X¹ is independently NH, NR¹, O, S, or SO₂; Y¹ is —CN, —Cl, —CHO, —COOH, —CONHR¹, —CON(R¹)₂, or —CO₂R¹; each of Z¹ and Z² is independently H, F, or C₁-C₆ alkyl; and each R¹ is independently C₁-C₆ alkyl;
comprising (a) admixing Compound B

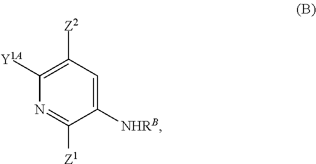

wherein Y^{1A} is —CN, —Cl, —CONHR¹, —CON(R¹)₂, or —CO₂R¹, R^{B} is hydrogen or —COOR⁴; and R⁴ is C₁₋₆alkyl; with a first transition metal catalyst and a boron-containing compound to form Compound C when R^{B} is hydrogen,

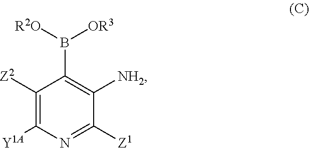

wherein each of R² and R³ is independently H or C₁-C₆ alkyl, or when taken together with the boron and oxygen atoms to which they are attached form a 5-, 6-, or 8-membered cyclic boronate, or to form Compound C′ when
R^{B} is

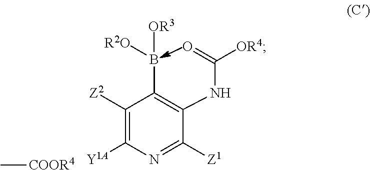

and optionally isolating Compound C or Compound C′, and
(b) admixing Compound C or Compound C′ with Compound D

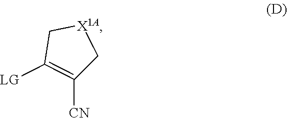

wherein X^{1A} is NR⁷, O, or S, and R⁷ is C₁-C₆alkyl, benzyl, or p-methoxybenzyl, and a second transition metal catalyst to form Compound A or a salt thereof, wherein LG is a leaving group. In embodiments where Y¹ of Compound A is CHO or COOH, the process can further comprise converting —CN, —Cl, —CONHR¹, —CON(R¹)₂, or —CO₂R¹ to CHO or COOH. In embodiments wherein X¹ of Compound A is NH, the process further comprises converting X^{1A} to NH.

The disclosure also provides processes for preparing Compound E, a stereoisomer thereof, a salt thereof, or a salt of a stereoisomer thereof,

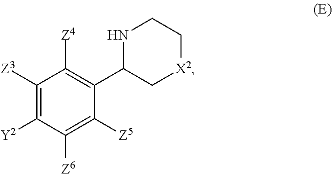

wherein X² is NR¹, O, or S, R¹ is C₁-C₆alkyl; Y² is H, C₁-C₆ alkyl, or C₁-C₆ haloalkyl; and each of Z³, Z⁴, Z⁵, and Z⁶ is independently H, C₁-C₆ alkyl, or chloride;
comprising admixing Compound F, or a salt thereof,

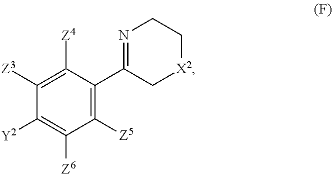

with an imine reductase (IRED) to form Compound E, a stereoisomer thereof, a salt thereof, or a salt of a stereoisomer thereof.

The disclosure further provides processes for preparing Compound I, a stereoisomer thereof, a salt thereof, or a salt of a stereoisomer thereof,

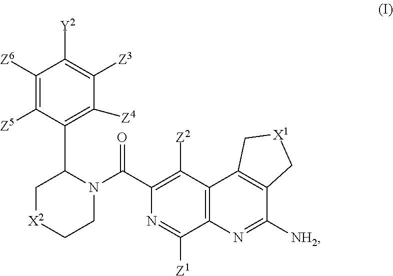

comprising admixing Compound A′, or a salt thereof, with Compound E, a stereoisomer thereof, a salt thereof, or a salt of a stereoisomer thereof, and a coupling agent to form Compound I, a stereoisomer thereof, a salt thereof, or a salt of a stereoisomer thereof,

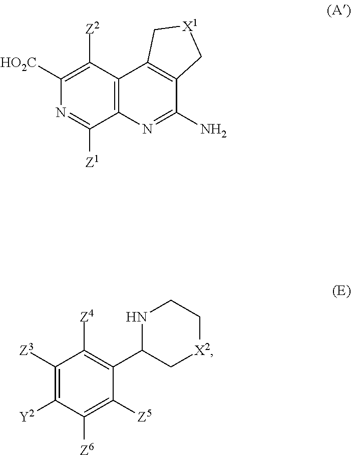

wherein X¹ is NH, NR¹, O, S, or SO₂, R¹ is C₁-C₆alkyl; X² is NR¹, O, or S; Y² is H, C₁-C₆ alkyl, or C₁-C₆ haloalkyl;
each of Z¹ and Z² is independently H, F, or C₁-C₆ alkyl; and
each of Z³, Z⁴, Z⁵, and Z⁶ is independently H, C₁-C₆alkyl, or chloride.

Also provided herein are compounds having a structure of

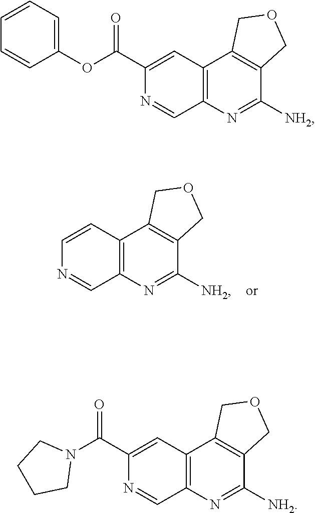 [SMILES: Nc1nc2cnc(C(=O)Oc3ccccc3)cc2c2c1COC2 ; Nc1nc2cnccc2c2c1COC2 ; Nc1nc2cnc(C(=O)N3CCCC3)cc2c2c1COC2]

### DETAILED DESCRIPTION

Provided here are processes for preparing various compounds useful as active pharmaceutical ingredients (API) and/or synthetic intermediates thereof.

In some embodiments, the disclosed processes are conducted in batch mode (i.e., “batch chemistry” or “fed-batch mode”). In other embodiments, the disclosed processes are conducted using continuous manufacturing processes (i.e., “flow chemistry” or “continuous chemistry”). As used herein, continuous manufacturing refers to an integrated system of unit of operations, with constant flow (steady or periodic). The disclosed processes utilizing continuous chemistry can provide the production of gram to metric ton quantities of active pharmaceutical ingredients (APIs). In some embodiments, the disclosed processes comprise a combination of steps that are conducted using batch chemistry and steps conducted using continuous chemistry.

In some embodiments, the disclosure provides processes for preparing Compound A, or a salt thereof, as shown in Scheme 1 and described herein.

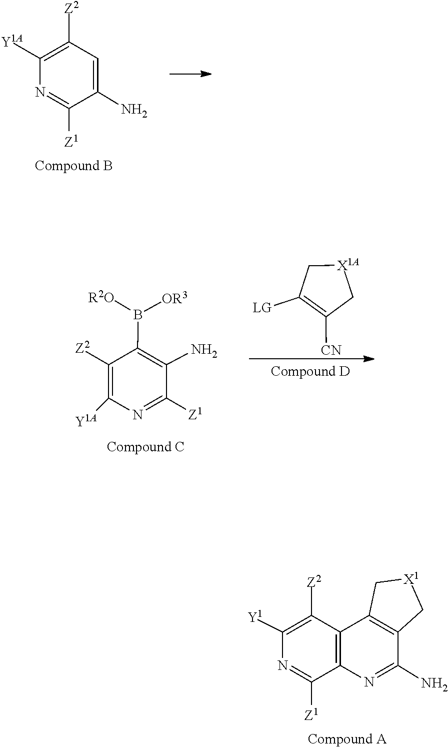

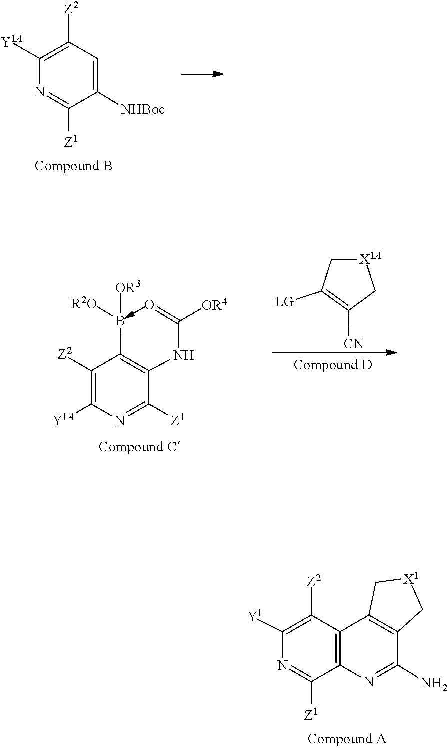

In some embodiments, the disclosure provides processes for preparing Compound E, a stereoisomer thereof, a salt thereof, or a salt of a stereoisomer thereof, as shown in Scheme 2 and described herein.

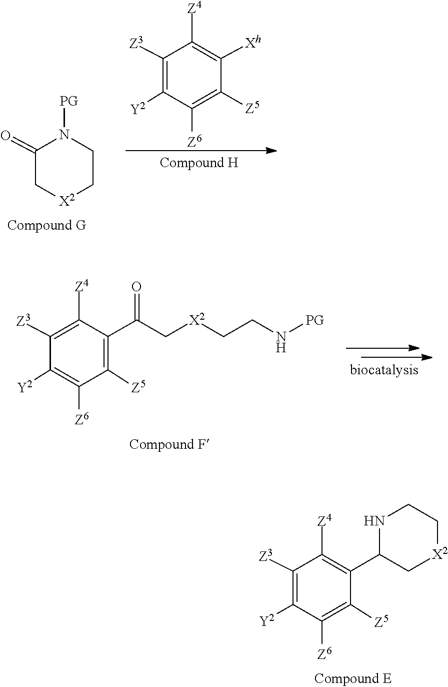

In some embodiments, the disclosure provides processes for preparing Compound I, a stereoisomer thereof, a salt thereof, or a salt of a stereoisomer thereof, as shown in Scheme 3 and described herein.

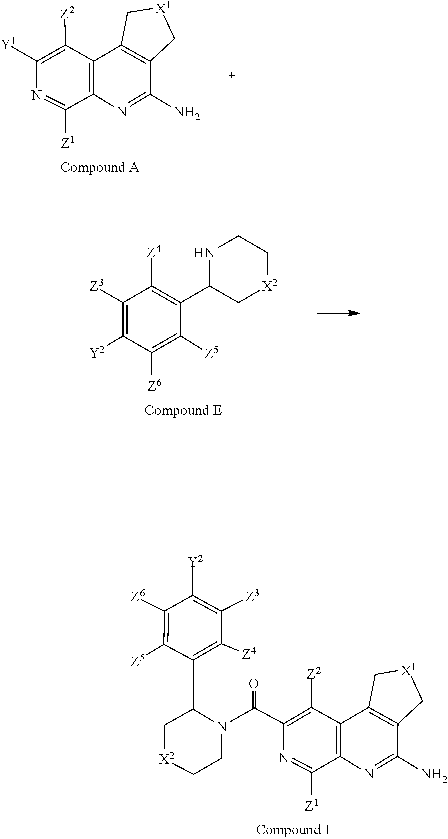

As described herein, the present disclosure provides processes for preparing Compound I, as well as starting materials/intermediates (e.g., Compounds A, A′, A1, C, C′, C1, C1-a, E, (S)-E, and salts thereof, stereoisomers thereof, and salts of stereoisomers thereof). In some embodiments, the disclosed processes advantageously provide Compound E in only three steps from commercially available materials while utilizing an enzymatic reaction. For example, the sequence of reactions described herein converting Compound F/F′ to Compound E comprise an enzymatic reduction, wherein a reduction catalyzed by an Imine Reductase (IRED) provides stereochemical control of the product (e.g., (S)-Compound E). Chiral control through enzyme-mediated processes are often highly selective, and in this instance, the disclosed processes provide the desired enantiomer in a stereochemical purity of greater than 99% enantiomeric excess (ee). Moreover, in some embodiments, the disclosed processes advantageously provide Compound A in two steps from commercially available raw materials in a process comprising a highly expedient and efficient one-step iridium C—H insertion/borylation followed by a palladium-mediated Suzuki reaction.

The disclosed processes advantageously provide Compound A in a one-step procedure using a transition metal (e.g., iridium) catalyzed/palladium catalyzed reaction. For example, in some embodiments, using an iridium C—H insertion/borylation reaction allows the process to start with the inexpensive and more readily available 5-aminopicolinonitrile rather than methyl 5-amino-4-bromopicolinate, which is used in other syntheses. Also, using the latter starting material requires isolating the boronate before the second Suzuki step resulting in lower yields. The disclosed processes provide either a one step or two step process with improved yields thereby enhancing efficiency and shortened manufacturing timelines and significant cost savings from the difference in starting materials needed.

In addition, the disclosed processes are cost-effective when compared to conventional processes. For example, Compound E requires long lead times for synthesis of large quantities over a multi-step process. In contrast, the disclosed processes provide Compound E in a much more efficient manner, requiring only three steps from commercially available starting material, two of which are performed in exclusively aqueous conditions, and therefore, cost effective manner.

In various embodiments, the disclosed processes for Compound E provide advantages over conventional processes which employ numerous steps, many having poor yields, requiring the use of expensive catalysts and chiral ligands, and use of high pressures. In contrast, the disclosed processes comprise a biocatalytic reduction, avoiding the need for high pressure and which can be performed in water and at lower temperatures (e.g., 20-50° C.). Furthermore, the biocatalytic process requires minimal unit operations—no extractions or distillations, only a pH adjustment and product filtration, resulting in fast batch cycle times.

The term “halide” or “halo” refers to F, Cl, Br, or I.

The term “alkyl” as used herein means a saturated straight or branched chain hydrocarbon. The term “cycloalkyl” refers to a non-aromatic carbon only containing ring system which is saturated, having three to six ring carbon atoms. Examples of C₁-C₆ alkyl groups include but are not limited to methyl, ethyl, isopropyl, n-propyl, isobutyl, n-butyl, sec-butyl, tert-butyl, isopentyl, n-pentyl, ne-opentyl, sec-pentyl, 3-pentyl, sec-isopentyl, active pentyl, isohexyl, n-hexyl, sec-hexyl, neohexyl, and tert-hexyl. Contemplated cycloalkyl groups include cyclopropyl, cyclobutyl, cyclopentyl, and cyclohexyl.

The term “haloalkyl” refers to an alkyl substituted with one or more (e.g., 1, 2, 3, 4, 5, or 6) halogen atoms. This term includes perfluorinated alkyl groups, such as —CF₃ and —CF₂CF₃.

### Compound A

In some embodiments, the disclosure provides processes for preparing Compound A, or a salt thereof,

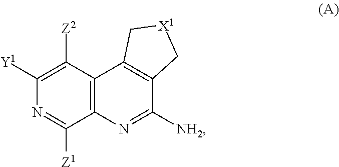

wherein X¹ is independently NH, NR¹, O, S, or SO₂; Y¹ is —CN, —Cl, —CHO, —COOH, —CONHR¹, —CON(R¹)₂, or —CO₂R¹; each of Z¹ and Z² is independently H, F, or C₁-C₆ alkyl; and each R¹ is independently C₁-C₆ alkyl.

In some embodiments, in conjunction with other above or below embodiments, X¹ is O.

In various embodiments, in conjunction with other above or below embodiments, Y¹ is —CN, —Cl, or —CO₂H. For example, in some embodiments, Y¹ is —CN, and in some embodiments, Y¹ is —CO₂H. Further, in some embodiments Y¹ is —Cl.

In some embodiments, in conjunction with other above or below embodiments, Z¹ and Z² are each H.

In some embodiments, in conjunction with other above or below embodiments, Compound A has a structure of A′:

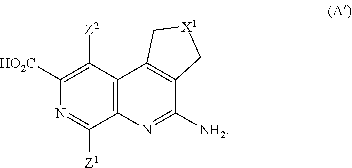

In some embodiments, Compound A′ is prepared by converting a Y¹ which is CHO, CN, CONHR¹, or CON(R¹)₂ to CO₂H. In some embodiments, Compound A′ has the following structure:

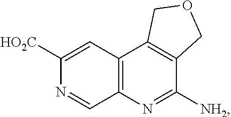 [SMILES: Nc1nc2cnc(C(=O)O)cc2c2c1COC2]

In some embodiments, in conjunction with other above or below embodiments, Compound A has a structure of A1:

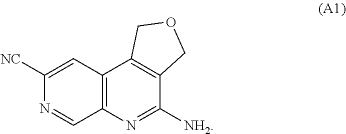 [SMILES: N#Cc1cc2c3c(c(N)nc2cn1)COC3]

In some embodiments, in conjunction with other above or below embodiments, Compound A has a structure of A2:

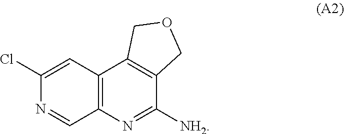 [SMILES: Nc1nc2cnc(Cl)cc2c2c1COC2]

### Compound E

In some embodiments, the disclosure provides processes for preparing Compound E, a stereoisomer thereof, a salt thereof, or a salt of a stereoisomer thereof,

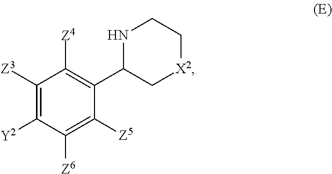

wherein X² is NR¹, O, or S, R¹ is C₁-C₆alkyl; Y² is H, C₁-C₆ alkyl, or C₁-C₆ haloalkyl; and each of Z³, Z⁴, Z⁵, and Z⁶ is independently H, C₁-C₆ alkyl, or chlorine.

As described herein, in some embodiments, Compound E is enriched in the (S)-stereoisomer:

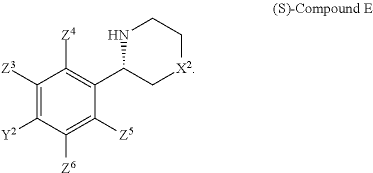

As used herein, “enriched in the (S)-stereoisomer” refers to the product (S)-stereoisomer having a higher stereochemical purity (as measured by percent enantiomeric excess) than the starting material. In some embodiments, the enantiomeric excess of the product (S)-Compound E may be 50% or more (e.g., 75%, 80%, 85%, 90% or 95% or more). In some embodiments, Compound F is converted to (S)-Compound E having greater than 99% ee. In some embodiments, Compound E is the (S)-Compound E having the following structure:

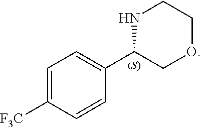

In some embodiments (S)-Compound E is a salt having the following structure:

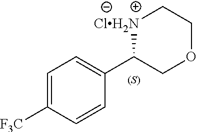

### Compound I

In various embodiments, the disclosure further provides processes for preparing Compound I, a stereoisomer thereof, a salt thereof, or a salt of a stereoisomer thereof:

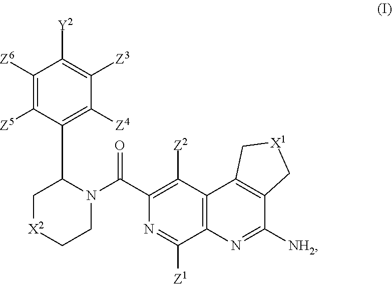

wherein X¹ is NH, NR¹, O, S, or SO₂; X² is NR¹, O, or S; R¹ is C₁-C₆alkyl; Y² is H, C₁-C₆ alkyl, or C₁-C₆ haloalkyl; each of Z¹ and Z² is independently H, F, or C₁-C₆ alkyl; and each of Z³, Z⁴, Z⁵, and Z⁶ is independently H, C₁-C₆alkyl, or chloride.

In some embodiments, Compound (I) is the (S)-stereoisomer of Compound I, or a salt thereof:

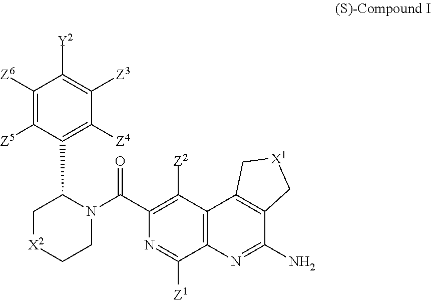

In some embodiments, Compound I has the following structure, or a salt thereof:

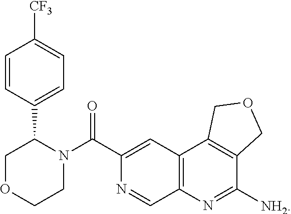 [SMILES: Nc1nc2cnc(C(=O)N3CCOC[C@@H]3c3ccc(C(F)(F)F)cc3)cc2c2c1COC2]

### Compound B

Compound B has the structure:

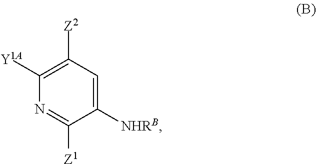

wherein each Z¹ and Z² are as defined for Compound A, R^{B} is hydrogen or —COOR⁴, and Y^{1A} is —CN, —Cl, —CONHR¹, CON(R¹)₂, or CO₂R¹, and R⁴ is C₁-C₆alkyl. In some embodiments, Y^{1A} is CN. In some embodiments, Y^{1A} is Cl. In some embodiments, R^{B} is tert-butyloxycarbonyl (Boc).

In some embodiments, Compound B has the structure

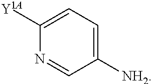

In some embodiments, Compound B has the structure B1:

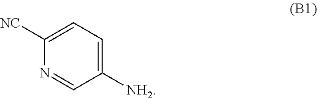

In some embodiments, Compound B has the structure of B1′:

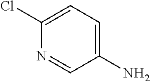

(B1′). In some embodiments, Compound B has the structure B2:

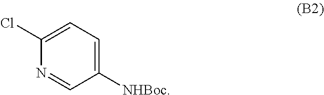 [SMILES: CC(C)(C)OC(=O)Nc1ccc(Cl)nc1]

### Compound C and Compound C′

In some embodiments, Compound C has the structure:

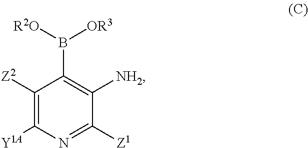

wherein each of R² and R³ is independently H or C₁-C₆ alkyl, or when taken together with the boron and oxygen atoms to which they are attached form a 5-, 6-, or 8-membered cyclic boronate; Y^{1A} is —CN, —Cl, —CONHR¹, —CON(R¹)₂, or —CO₂R¹, R¹ is C₁-C₆alkyl, and each of Z¹ and Z² is independently H, F, or C₁-C₆ alkyl.

In some embodiments, Compound C has a structure of C1:

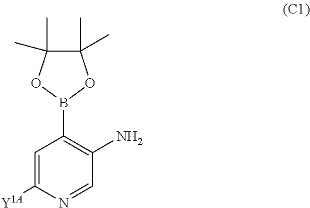

In some embodiments, Compound C has a structure of C1-a:

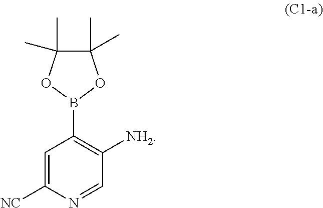 [SMILES: CC1(C)OB(c2cc(C#N)ncc2N)OC1(C)C]

In some embodiments, Compound C′ has a structure:

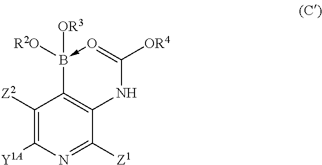

wherein each of R² and R³ is as defined for Compound C and R⁴ is C₁-C₆alkyl. In some cases, Compound C′ has a structure of:

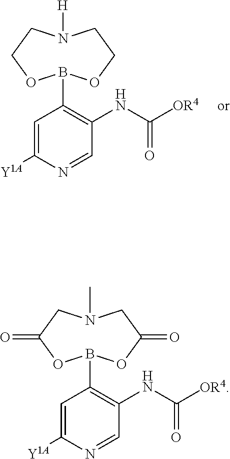

In some embodiments, Compound C′ has a structure of C′-2:

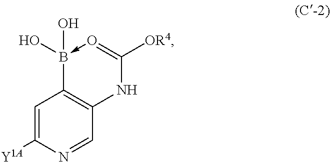

In some embodiments, Compound C′ has a structure of C′-3:

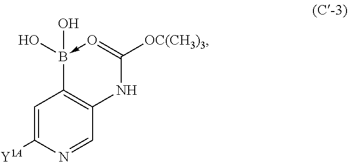

In some embodiments, Compound C′ has a structure of C′-4:

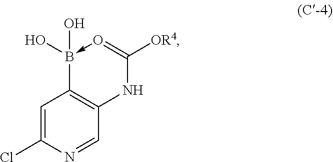

In some embodiments, Compound C′ has a structure of C′-5, C′-6, or C′-7:

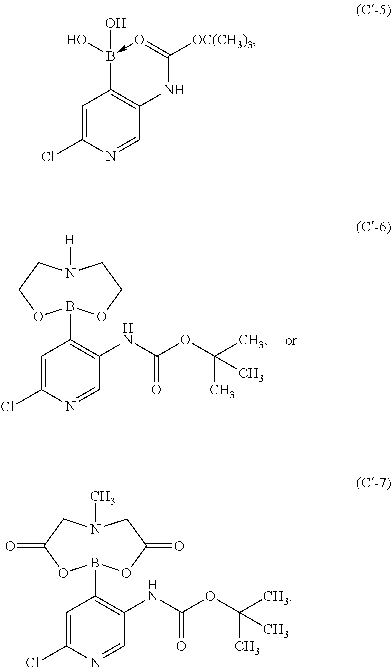

In some cases, Compound C′ has a structure of C′-5. In some cases, Compound C′ has a structure of C′-6. In some cases, Compound C′ has a structure of C′-7.

### Compound D

In some embodiments, Compound D has the following structure:

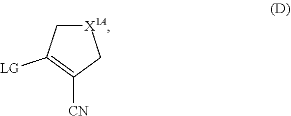

wherein X^{1A} is NR⁷, O, or S, and R⁷ is C₁-C₆alkyl, benzyl, or p-methoxybenzyl and LG is a leaving group. In embodiments wherein X¹ of Compound A is different than X^{1A} of Compound D, the processes can further comprise converting X^{1A} to X¹. For example, in embodiments where X¹ of Compound A is NH, the process further comprises converting X^{1A} to NH.

The leaving group can be any suitable leaving group. Specific contemplated leaving groups include, for example, a sulfonate ester, a sulfamate, or a halide. In some embodiments, the leaving group is tosyl, mesyl, nosyl, or triflyl. In some embodiments, the leaving group is halide (e.g., F, Cl, Br, or I). In some cases, the halide leaving group is Cl, Br, or I.

In some embodiments, Compound D has a structure of D1:

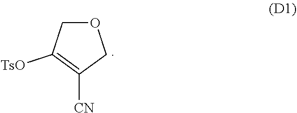 [SMILES: Cc1ccc(S(=O)(=O)OC2=C(C#N)COC2)cc1]

### Compound F

In some embodiments, Compound F has the following structure:

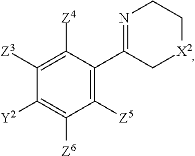

wherein each of X², Y², Z³, Z⁴, Z⁵, and Z⁶ is as defined for Compound E.

In some embodiments, Compound F has a structure of FR:

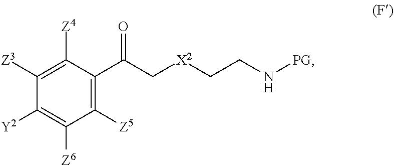

wherein PG is a protecting group.

The protecting group is any suitable protecting group for an amine nitrogen. In some embodiments, the protecting group is selected from the group consisting of tert-butyloxycarbonyl (Boc), benzyloxycarbonyl (Cbz), and trimethylsilyl (TMS). In some embodiments, the protecting group is Boc. In some embodiments, the protecting group is Cbz. In some embodiments, the protecting group is TMS.

In some embodiments, Compound F has the following structure:

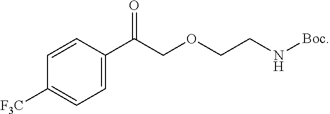 [SMILES: CC(C)(C)OC(=O)NCCOCC(=O)c1ccc(C(F)(F)F)cc1]

### Compound G

In some embodiments, the disclosed processes comprise forming Compound F or Compound F′ by admixing Compound G or a salt thereof, Compound H, and an organometallic reagent or magnesium metal. In some embodiments, Compound G has the following structure:

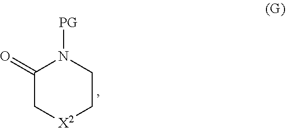

wherein X² is as defined for Compound E and PG is a protecting group as defined for Compound F′. In some embodiments, the protecting group of Compound G is Boc.

In some embodiments, Compound G has the following structure:

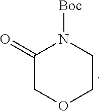 [SMILES: CC(C)(C)OC(=O)N1CCOCC1=O]

### Compound H

In some embodiments, Compound H has the following structure:

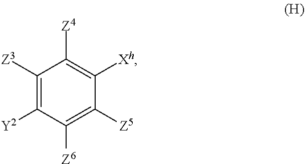

wherein Y² and each of Z³, Z⁴, Z⁵, and Z⁶ is as defined for Compound E and X^{h} is Cl, Br, or I.

In some embodiments, X^{h} is I. In some embodiments X^{h} is Br. In some embodiments Y² is CF₃. In some embodiments, each of Z³, Z⁴, Z⁵, and Z⁶ is H.

In some embodiments, Compound H has the following structure:

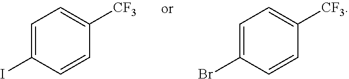 [SMILES: FC(F)(F)c1ccc(I)cc1 ; FC(F)(F)c1ccc(Br)cc1]

### Processes for Preparing Compounds A and B

The disclosed processes for preparing Compound A or a salt thereof comprise (a) admixing Compound B with a first transition metal catalyst and a boron-containing compound to form Compound C and (b) admixing Compound C with Compound D and a second transition metal catalyst to form Compound A, or a salt thereof. In some embodiments, Compound C is Compound C1 or C1-a.

Compound B is admixed with a suitable amount of the first transition metal catalyst and boron-containing compound to form Compound C. In some embodiments, Compound B is admixed with less than one equivalent (eq) of the boron-containing compound (e.g., 0.5 eq of the boron-containing compound) to form Compound C.

Compound C is admixed with a suitable amount of Compound D and a second transition metal catalyst to form Compound A, or a salt thereof. In some embodiments, Compound C is admixed with at least one equivalent of Compound D (e.g., 1.0, 1.1, 1.2, 1.3, 1.4, or 1.5 eq or more of Compound D). In some embodiments, X^{1A} is converted to X¹.

In some embodiments, the process further comprises isolating Compound C (e.g., via crystallization or chromatography). In some embodiments, the process for preparing Compound A or a salt thereof is conducted in a vessel without isolating Compound C (e.g., a “one-pot” process). In these instances, Compound C is carried forward directly to step (b) without isolation.

In some embodiments, the disclosed processes further comprise preparing Compound B (e.g., Compound B1) or a salt thereof using biocatalysis (e.g., a biocatalytic reduction). Aspects of the disclosed processes are described in Bornadel et al. “Process Development and Protein Engineering Enhanced Nitroreductase-Catalyzed Reduction of 2-Methyl-5-nitropyridine.” Org. Process Res. Dev. 25, 3, (2021): 648-653, the disclosure of which is incorporated herein by reference.

In some embodiments, the disclosed processes comprise preparing Compound B1 or Compound B1′ from 2-cyano-5-nitropyridine or 2-chloro-5-nitropyridine

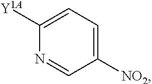

or a salt thereof.

By way of example, some embodiments of the disclosed processes comprise admixing 2-cyano-5-nitropyridine or 2-chloro-5-nitropyridine or a salt thereof with a nitroreductase in a solvent to form Compound B1 or Compound B1′, or a salt thereof.

The nitroreductase can be any suitable nitroreductase capable of transforming 2-cyano-5-nitropyridine, 2-chloro-5-nitropyridine, or salt thereof to Compound B1 or a salt thereof. Suitable nitroreductases are commercially available (e.g., Johnson Matthey (London, U.K.)). Suitable nonlimiting examples of nitroreductases include NR-17, NR-X4-mut2, NR-X4-mut10, NR-X18, NR-X27, NR-X30, NR-X32, NR-X36, NR-X39, NR-X41, NR-X53, NR-X54, and a combination thereof, commercially available from Johnson Matthey. In some embodiments, the nitroreductase is NR-17 or NR-X36.

A suitable amount of nitroreductase is employed in the disclosed processes to provide Compound B1 or Compound B1′, or a salt thereof. If too little NR-17 or NR-X36 is present, the enzymatic reaction may not proceed at a suitable rate. In contrast, if too much NR-17 or NR-X36 is present, the reaction will not be cost efficient and may lead to undesirable side-products. In some embodiments, NR-17 is present in an amount of 0.1-10 wt %, based upon 2-cyano-5-nitropyridine (e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 wt % NR-17 based upon 2-cyano-5-nitropyridine). Thus, the NR-17 or NR-X36 can be present in an amount bounded by and including any of the aforementioned values, for example, 0.1-10, 0.2-9.9, 0.3-9.8, 0.4-9.7, 0.5-9.6, 0.6-9.5, 0.7-9.4, 0.8-9.3, 0.9-9.2, or 1-9.1 wt % based upon 2-cyano-5-nitropyridine or 2-chloro-5-nitropyridine (e.g., 1-10, 1-9, 2-9, 2-8, 3-8, 3-7, 4-7, 4-6, 5-6 wt % based upon 2-cyano-5-nitropyridine). In some embodiments, NR-17 or NR-36 is present in an amount of 5-7 wt % based upon 2-cyano-5-nitropyridine or 2-chloro-5-nitropyridine.

In some embodiments, the biocatalytic reduction of 2-cyano-5-nitropyridine, 2-chloro-5-nitropyridine or salt thereof in the disclosed processes further comprises admixing 2-cyano-5-nitropyridine, 2-chloro-5-nitropyridine or salt thereof and the nitroreductase in the presence of one or more of a glucose dehydrogenase (GDH), a third transition metal catalyst, a co-factor, a reductant, or a buffer. In some embodiments, the disclosed processes comprise admixing 2-cyano-5-nitropyridine, 2-chloro-5-nitropyridine or salt thereof and the nitroreductase in the presence of a glucose dehydrogenase (GDH), a third transition metal catalyst, a co-factor, a reductant, and a buffer. In embodiments comprising a co-factor, a reductant, and a GDH, the co-factor (e.g., NADPH) is regenerated via the catalytic oxidation of the reductant (e.g., glucose) by GDH.

### Third Transition Metal Catalyst

As described herein, some embodiments of the disclosed process comprise admixing 2-cyano-5-nitropyridine, 2-chloro-5-nitropyridine, or salt thereof and a nitroreductase in the presence of a third transition metal catalyst. In some embodiments, the third transition metal catalyst comprises vanadium, iron, copper, or a combination thereof. In some embodiments, the third transition metal catalyst comprises vanadium. A suitable form of vanadium is ammonium metavanadate (NH₄VO₃) or vanadium (V) oxide (e.g., vanadium(IV) oxide and/or vanadium(V) oxide). In some embodiments, the third transition metal catalyst is ammonium metavanadate (NH₄VO₃) or vanadium pentoxide (V₂O₅).

A suitable amount of third transition metal catalyst is employed in the disclosed processes. If too little third transition metal catalyst is present, the enzymatic reaction may not proceed at a suitable rate or it may lead to undesirable side-product formations. In contrast, if too much third transition metal catalyst is present, the reaction will not be cost efficient and may lead to undesirable side-products. In some embodiments, the third transition metal catalyst is present in an amount of 0.01-2 eq, based upon 2-cyano-5-nitropyridine or 2-chloro-5-nitropyridine (e.g., 0.01, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2 eq based upon 2-cyano-5-nitropyridine). In some embodiments, the third transition metal catalyst is present in an amount of 0.05-0.2 eq, based upon 2-cyano-5-nitropyridine or 2-chloro-5-nitropyridine (e.g., 0.05, 0.08, 0.1, 0.15, or 0.2 eq third transition metal catalyst based upon 2-cyano-5-nitropyridine or 2-chloro-5-nitropyridine). Thus, the third transition metal catalyst can be present in an amount bounded by and including any of the aforementioned values (e.g., 0.01-2, 0.1-1.9, 0.2-1.8, 0.3-1.7, 0.4-1.6, 0.5-1.5, 0.6-1.4, 0.7-1.3, 0.8-1.2, or 0.9-1.1 eq based upon 2-cyano-5-nitropyridine or 0.05-0.2, 0.08-0.15 eq based upon 2-cyano-5-nitropyridine). In some embodiments, the third transition metal catalyst is present in an amount of 0.1 eq based upon 2-cyano-5-nitropyridine or 2-chloro-5-nitropyridine. In some embodiments, the third transition metal catalyst is present in an amount of 2 eq based upon 2-cyano-5-nitropyridine or 2-chloro-5-nitropyridine.

### Glucose Dehydrogenase (GDH)

As described herein, some embodiments of the disclosed process comprise admixing 2-cyano-5-nitropyridine or 2-chloro-5-nitropyridine and a nitroreductase in the presence of a glucose dehydrogenase (GDH). GDH is present in some embodiments of the disclosed process to facilitate the regeneration of the co-factor.

Suitable glucose dehydrogenases are commercially available (e.g., Johnson Matthey (London, U.K. and Codexis (Redwood City, CA)). Suitable nonlimiting examples of glucose dehydrogenase include, GDH-5, GDH-8, GDH-101, GDH-105, CDX-901, and a combination thereof. In some embodiments, the glucose dehydrogenase is GDH-101. By way of example, GDH-101 is commercially available from Johnson Matthey, and GDH-105 and CDX-901 are commercially available from Codexis.

A suitable amount of glucose dehydrogenase is employed in the disclosed processes. If too little glucose dehydrogenase is present, the enzymatic reaction may not proceed at a suitable rate. In contrast, if too much glucose dehydrogenase is present, the reaction will not be cost efficient and may lead to undesirable side-products. In some embodiments, glucose dehydrogenase is present in an amount of 0.1-25 wt %, based upon 2-cyano-5-nitropyridine or 2-chloro-5-nitropyridine (e.g., 0.1, 0.5, 1, 2, 3, 4, 5, 6, 7, 8, 9, 10, 11, 12, 13, 14, 15, 16, 17, 18, 19, 20, 21, 22, 23, 24, or 25 wt %, based upon 2-cyano-5-nitropyridine). Thus, the glucose dehydrogenase can be present in an amount bounded by and including any of the aforementioned values (e.g., 0.1-25, 0.5-25, 1-24, 2-23, 3-22, 4-21, 5-20, 6-19, 7-18, 8-17, 9-16, 10-15, 11-14, or 12-13 wt %, based upon 2-cyano-5-nitropyridine). In some embodiments, glucose dehydrogenase is present in an amount of 1 wt %, based upon 2-cyano-5-nitropyridine.

### Co-Factor

As described herein, some embodiments of the disclosed process comprise admixing 2-cyano-5-nitropyridine or 2-chloro-5-nitropyridine and a nitroreductase in the presence of a co-factor. As is understood, the co-factor facilitates the biocatalytic reduction reaction catalyzed by the nitroreductase. Suitable nonlimiting examples of co-factors include, nicotinamide adenine dinucleotide (NAD+), dihydronicotinamide adenine dinucleotide (NADH), nicotinamide adenine dinucleotide phosphate (NADP+), dihydronicotinamide adenine dinucleotide phosphate (NADPH), a salt of NADPH, and a combination thereof. In some embodiments, the co-factor is NADP+.

A suitable amount of co-factor is employed in the disclosed processes. If too little co-factor is present, the enzymatic reaction may not proceed at a suitable rate. In contrast, if too much co-factor is present, the reaction will not be cost efficient and may lead to undesirable side-products. In some embodiments, co-factor is present in an amount of 0.5-20 wt %, based upon 2-cyano-5-nitropyridine or 2-chloro-5-nitropyridine (e.g., 0.5, 0.6, 0.7, 0.8. 0.9, 1, 2, 3, 4, 5, 6, 7, 8, 9, 10, 11, 12, 13, 14, 15, 16, 17, 18, 19, or 20 wt %, based upon 2-cyano-5-nitropyridine). Thus, the co-factor can be present in an amount bounded by and including any of the aforementioned values (e.g., 0.5-20, 0.6-19, 0.7-18, 0.8-17, 0.9-16, 1-15, 2-14, 3-13, 4-12, 5-11, 6-10, or 7-9 wt % co-factor, based upon 2-cyano-5-nitropyridine). In some embodiments, co-factor is present in an amount of 0.7 wt %, based upon 2-cyano-5-nitropyridine.

### Reductant

As described herein, some embodiments of the disclosed process comprise admixing 2-cyano-5-nitropyridine or 2-chloro-5-nitropyridine and a nitroreductase in the presence of a reductant. The reductant facilitates the regeneration of the co-factor. In some embodiments, the reductant is glucose.

A suitable amount of reductant is employed in the disclosed processes. If too little reductant is present, the enzymatic reaction may not proceed at a suitable rate. In contrast, if too much reductant is present, the reaction will not be cost efficient and may lead to undesirable side-products. In some embodiments, reductant is present in an amount of 3-5 eq, based upon 2-cyano-5-nitropyridine or 2-chloro-5-nitropyridine (e.g., 3.0, 3.1, 3.2, 3.3, 3.4, 3, 5, 3.6, 3.7, 3.8, 3.9, 4.0, 4.1, 4.2, 4.3, 4.4, 4.5, 4.6, 4.7, 4.8, 4.9, or 5.0 eq, based upon 2-cyano-5-nitropyridine or 2-chloro-5-nitropyridine). Thus, the reductant can be present in an amount bounded by and including any of the aforementioned values (e.g., 3.0-5.0, 3.5-4.5, or 3.0-4.0 eq reductant, based upon 2-cyano-5-nitropyridine or 2-chloro-5-nitropyridine). In some embodiments, reductant is present in an amount of 3.1 eq, based upon 2-cyano-5-nitropyridine or 2-chloro-5-nitropyridine.

### Buffers for Preparing Compound B

In some embodiments, the disclosed process are conducted in the presence of suitable buffer. Suitable buffers include those capable of maintaining a pH of 7 to 8 (e.g., 7.0, 7.1, 7.2, 7.3, 7.4, 7.5, 7.6, 7.7, 7.8, 7.9, or 8.0). In some embodiments, buffer maintains a pH of 7.2 to 7.5. In some embodiments, the buffer comprises a tricine buffer, a potassium phosphate buffer, 4-(2-hydroxyethyl)-1-piperazineethanesulfonic acid (HEPES), tris(hydroxymethyl)aminomethane (Tris), or a combination thereof. In some embodiments the buffer is a potassium phosphate buffer.

The buffer is present in any suitable amount. If the amount of buffer is too low, then the pH of the reaction will not be maintained properly (e.g., pH 7-8). In contrast, if the amount of buffer is too high, then the reaction will not be cost efficient and may lead to undesirable side-products. In some embodiments, the buffer is present in an amount of 80-95% (v/w) (e.g., 80-90%, 80-85%, 85-95%, 85-90%, or 90-95% (v/w)). In some embodiments, the buffer is present in an amount of 92% (v/w). In some embodiments, the buffer is present in an amount of 100-250 mM, (e.g., 100, 125, 150, 175, 200, 225, or 250 mM).

### Solvents for Preparing Compound B

The processes disclosed herein for preparing Compound B are conducted in a suitable solvent. Suitable nonlimiting examples of organic co-solvents include ethanol, isopropyl alcohol, tert-butyl alcohol, tetrahydrofuran, 2-methyltetrahydrofuran, methyl tert-butyl ether (MTBE), toluene, isoamyl acetate, tert-butyl acetate, cyclopentyl methyl ether, dimethylacetamide, acetone, dimethyl carbonate, acetonitrile, and a combination thereof. In some embodiments, the admixing of 2-cyano-5-nitropyridine or salt thereof with the nitroreductase is conducted in a solvent comprising water, dimethylsulfoxide (DMSO), toluene, MTBE, isopropyl alcohol, isopropyl acetate, or a combination thereof. In some embodiments, the admixing of 2-cyano-5-nitropyridine or salt thereof with the nitroreductase is conducted in a solvent comprising water, dimethylsulfoxide (DMSO), or a combination thereof. In some embodiments, the solvent comprises DMSO. Without wishing to be bound to any particular theory, DMSO functions as an organic co-solvent. Typically, DMSO can be present in an amount of 0.5-20 volumes (e.g., 0.5, 1, 2, 3, 4, 5, 6, 7, 8, 9, 10, 11, 12, 13, 14, 15, 16, 17, 18, 19, or 20 volumes, based upon 2-cyano-5-nitropyridine). In some embodiments, the solvent comprises 0.5 volumes of DMSO, based upon 2-cyano-5-nitropyridine.

### Temperature

The processes disclosed herein for preparing Compound B are conducted at a suitable temperature, typically at a temperature of 20-50° C. In some embodiments, 2-cyano-5-nitropyridine or salt thereof is admixed with the nitroreductase at a temperature of 32-38° C. (e.g., 35-38° C.).

### Fed-Batch Mode

In some embodiments, the disclosed processes for preparing Compound B are conducted in fed-batch mode. In illustrative embodiments conducted in fed-batch mode, 2-cyano-5-nitropyridine or 2-chloro-5-nitropyridine can be added to the reaction mixture containing the other components via continuous addition (e.g., a syringe pump at a constant flow rate). Conducting the disclosed processes in fed-batch mode provides advantages such as, for example, lowering the required amounts of nitroreductase, third transition metal catalyst, co-factor, and solvent needed to conduct the reaction. For example, in some embodiments the amount of total enzyme (NR and GDH) required is reduced by about 70%; the amount of third transition metal catalyst (e.g., NH₄VO₃) required is reduced by about 88%; the amount of co-factor (e.g., NADPH) required is reduced by about 95%; and/or the amount of solvent required is reduced by about 97%.

The processes for preparing Compound A or a salt thereof can be conducted in either batch mode or continuous mode.

The disclosed processes provide Compound A or a salt thereof in a suitable yield. In some embodiments, Compound A or a salt thereof is prepared in an overall yield of 40% or more, based upon Compound B (e.g., in a yield of 40, 45, 50, 55, 60, 65, 70, 75, 80, 85, 90, or 95% or more, based upon Compound B). In some embodiments wherein Compound C is isolated prior to reaction with Compound D (e.g., a “two-pot” process”), Compound A or a salt thereof is obtained in an overall yield of at least 40% or more, e.g., 40-60%, 45-60%, 50-60%, 50-55%, or 55-60%, based upon Compound B. In some embodiments wherein Compound C is not isolated prior to reaction with Compound D (e.g., an “one-pot” process), Compound A is obtained in an overall yield of 50% or more, for example, 60% or more. In some embodiments, Compound A is obtained from an one-pot process in an overall yield of 60-95%, 60-80%, or 60-70%.

In some embodiments, Compound A1, or a salt thereof, is converted to Compound A′, or a salt thereof. In these embodiments, Compound A1 is converted to Compound A′ using any suitable reactions conditions for converting the —CN functional group of Compound A1 to the —CO₂H functional group of Compound A′. In some embodiments, the conversion of Compound A1 to Compound A′ is conducted using basic conditions to hydrolyze the —CN functional group. In some embodiments, Compound A1 or a salt thereof is converted to Compound A′ or a salt thereof in a chemical yield of 90% or more (e.g., 91%, 92%, 93%, 94%, 95%, 96%, 97%, 98%, 99% or more). In some embodiments, Compound A1 is converted to Compound A′ using basic hydrolysis conditions.

### Solvent and Temperature to Form Compound C or Compound C′

The admixing of Compound B with a first transition metal catalyst and a boron-containing compound is conducted in a suitable solvent. In some cases, the solvent is an aprotic solvent. Illustrative aprotic solvents include, for example, tetrahydrofuran, 1,4-dioxane, 2-methyl tetrahydrofuran, cyclopentyl methyl ether (CPME), and toluene. In some embodiments, the solvent is tetrahydrofuran (THF).

The admixing of Compound B with a first transition metal catalyst and a boron-containing compound is conducted at a suitable temperature. In some cases, the reaction is conducted at a temperature of about 50-100° C. (e.g., 50, 55, 60, 65, 70, 75, 75, 80, 85, 90, 95, or 100° C.). In some embodiments, the temperature is 55-95° C., 60-90° C., 65-85° C., 70-80° C., or 75° C. For example, in some embodiments, Compound B is admixed with a first transition metal catalyst and a boron-containing compound at approximately 65° C. In some embodiments, Compound B, a first transition metal catalyst, and a boron-containing compound are added together in a reaction vessel at a lower temperature (e.g., 25-35° C.) prior to admixing at a higher temperature (e.g., 60-65° C.). In some embodiments, the reaction mixture is allowed to cool to a lower temperature (e.g., 40-45° C.) before the reaction mixture is quenched.

### Boron-Containing Compound

The boron-containing compound is any suitable boron compound compatible with the desired borylation reaction under the desired reaction conditions. In some embodiments, the boron-containing compound is 4,4,4′,4′,5,5,5′,5′-octamethyl-2,2′-bi(1,3,2-dioxaborolane) or 4,4,5,5-tetramethyl-1,3,2-dioxaborolane.

### First Transition Metal Catalyst

The first transition metal catalyst is any suitable transition metal catalysts capable of affecting the borylation desired transformations. As such, the first transition metal catalyst is any suitable transition metal catalyst capable of catalyzing the conversion of Compound B to Compound C or C′. A contemplated first transition metal catalyst comprises iridium. In some embodiments, the first transition metal catalyst is [Ir(OMe)(COD)]₂ or [Ir(Cl)(COD)]₂. As is understood, these iridium catalysts are used in conjunction with organic ligands to facilitate the desired reactivity. Suitable ligands include, for example, 4,4′ di-tert-butyl-2,2′-biypridine (diby), 3,4,7,8-tetramethyl-1,10,phenanthroline, and 1,10-phenanthroline.

The first transition metal catalyst is used in a suitable amount. If too little catalyst is used, then the desired reaction rate may not be obtained. Conversely, if too much catalyst is used, undesired side products may be obtained, and/or the cost of the reaction is unnecessarily high. In some embodiments, the first transition catalyst is present in an amount of 0.3 to mol %, based upon Compound B (e.g., 0.3, 0.4, 0.5, 0.6, 0.7, 0.8, 0.9, 1, 1.25 1.5, 1.75, 2, 2.25, 2.5, 2.75, 3, 3.25, 3.5, 3.75, 4, 4.25, 4.5, 4.75, or 5 mol % based upon Compound B). In some embodiments, the first transition metal catalyst is present in an amount of 1.5 mol % (as a dimeric complex) based upon Compound B. As is understood, the metal catalyst without ligand can exist as a dimer such that after adding the ligand, the first transition metal-ligand catalyst is present in an amount of 3 mol % based upon Compound B. In some embodiments, the first transition metal catalyst is 1.5 mol % [Ir(OMe)(cod)]₂-3% dibpy. In some embodiments, the first transition metal catalyst is prepared by mixing a solution of the boron containing compound (e.g., bis(pinacolato)diboron) (0.5 eq dimer; 1 eq borane), a ligand (0.03 eq), and an iridium containing compound (0.015 eq). In some embodiments, an excess of the boron containing compound is used. For example, in some embodiments, 1.5 eq of pinacolborane is added to form the N-boronate derivative, followed by addition of the first transition metal catalyst and bis(pinalcolato)diborane. In some embodiments, 2 eq or more of pinacol borane is added followed by the first transition metal catalyst. Typically, in embodiments wherein 2 eq or more of pinalcol borane is added, bis(pinacolato)diboron need not be added to the reaction mixture. In some embodiments, Compound B is added as a solution of the first transition metal-ligand catalyst and boron containing compound.

### Second Transition Metal Catalyst

As described herein, the disclosed processes for preparing Compound A also comprise admixing Compound C or C′ with Compound D and a second transition metal catalyst. In some embodiments, the admixing of Compound C or C′ with Compound D and a second transition metal catalyst is conducted in a solvent comprising a mixture of an organic solvent (e.g., THF) and water. As with the first transition metal catalyst, the second transition metal catalyst is any suitable catalyst capable of affecting the coupling of Compound C or C′ with Compound D under the desired conditions. Contemplated second transition metal catalysts comprise a palladium catalyst or a nickel catalyst. In some embodiments, the second transition metal catalyst is dichloro[9,9-dimethyl-4,5-bis(diphenylphosphino)xanthene]palladium(II). In some embodiments, the second transition metal catalyst is 1,4-bis(diphenylphosphino)butane-palladium(II) chloride, bis(1,5-cyclooctadiene)nickel(0) with tri-n-butylphosphonium tetrafluoroborate, or [(N,N,N′,N′-tetramethylethane-1,2-diamine)nickel(ortho-tolyl)chloride] complex. In some embodiments, the second transition metal catalyst is chloro(2-methylphenyl)(N,N,N′,N′-tetramethyl-1,2-ethylenediamine)nickel(II) with tri-n-butylphosphine. In some embodiments, reducing additives like n-hexylmagnesium chloride, methylmagnescium chloride, manganese or zinc are added.

Similar to the first transition metal catalyst, the second transition metal catalyst is used in a suitable amount. In some embodiments, the second transition metal catalyst is present in an amount of 1 to 10, or 1 to 5, mol %, based upon Compound B. In some embodiments, the second transition metal catalyst is prepared by mixing a phosphine ligand and Pd catalyst in an organic solvent.

### Alternative Synthetic Scheme for Compound C′

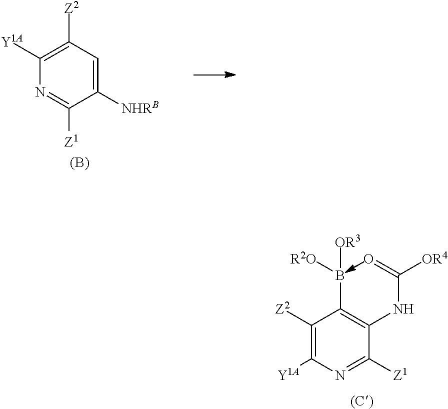

Compound C′ can be prepared as generally outlined in the above scheme.

Admixing Compound B first with a metal-amide base (e.g., where the metal is methylmagnesium chloride, ethylmagnesium chloride, isopropylmagnesium chloride, n-hexylmagnesium chloride, methylmagnesium bromide, ethylmagnesium bromide, isopropylmagnesium bromide, n-hexylmagnesium bromide, n-butyllithium, or tert-butyllithium; and the amide is 2,2,6,6-tetramethylpiperidine, diisopropylamine), then a boron-containing compound (trimethylborate, triethylborate, triisopropylborate, 2-methoxy-4,4,5,5-tetramethyl-1,3,2-dioxaborolane, or 2-ethoxy-4,4,5,5-tetramethyl-1,3,2-dioxaborolane) and then optionally treated with water or diols (diethanolamine) or diacids (methyliminodiacetic acid) to form Compound C′. Compound C′ can be isolated or used directly in the next step. Preparation of Compound C′ in this manner provides a number of advantages, including cost and sustainability using metal-amide bases to promote this transformation (instead of precious metal catalysts). Further, isolation of a crystalline boronate ester can provide better purity and yield.

In some embodiments, preparation of Compound C′ comprises using methylmagnesium chloride (amounts from 2.0 to 5.0; or more specifically 3.6 molar equivalents), and/or 2,2,6,6-tetramethylpiperidine (amounts from 1.0 to 4.0, or more specifically 3.6 molar equivalents), and/or triethylborate (amounts from 2.0 to 5.0; or more specifically 3.8 molar equivalents). In some cases, reaction of Compound B to form Compound C′ is performed in a solvent such as ether-containing solvents (tetrahydrofuran, 2-methyltetrahydrofuran, 1,2-dimethoxyethane, tert-butyl methyl ether, isopropyl ether). In some cases, 7.5 L/kg tetrahydrofuran and 12.5 L/kg 1,2-dimethoxyethane are used. In some cases, treatment with a diol such as diethanolamine to form the Compound C′-6 is contemplated.

In some cases, Compound C′ is prepared as shown in the below scheme:

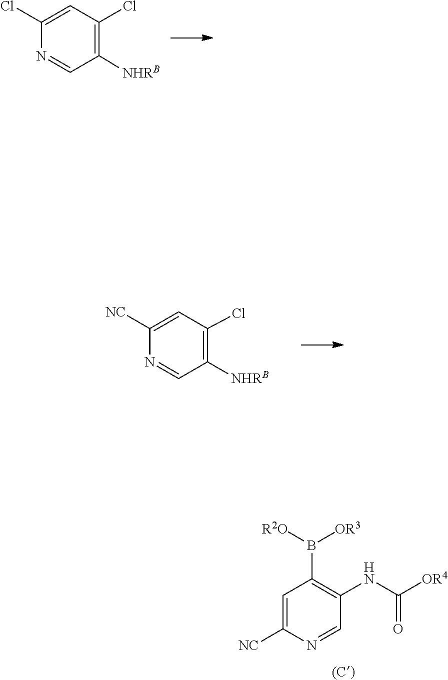

As shown in the above scheme, Compound C′ can be prepared by admixing the dichloro-pyridyl compound with a metal catalyst to form the cyano-chloride pyridyl compound. For example, 1.5% molar equivalents of (tris)dibenzylideneacetonepalladium(0) and 3.0% molar equivalents of 1,1′-bis(di-tert-butylphosphino)ferrocene and 0.65 molar equivalents of zinc(II) cyanide or potassium ferrocyanide can be mixed with the dichloro-pyridyl compound in the presence of 20 molar equivalents of zinc metal, in solvents such as N,N-dimethylacetamide (e.g., 9 L/kg) and tetrahydrofuran (e.g., 1 L/kg) at 70° C. to produce the cyano-chloro-pyridyl compound shown above. The cyano-chloro-pyridyl compound can then be admixed with palladium(II) acetate (e.g., 2.5% molar equivalents) and 2-dicyclohexylphosphino-2′,6′-dimethoxybiphenyl (also known as SPhos. e.g. 5.0 molar equivalents) in the presence of a boron source like bis(pinacolato)diboron (e.g., 1.2 molar equivalents) and in the presence of a base (e.g., 2 molar equivalents of potassium acetate) in an ether solvent (e.g., 2-methyltetrahydrofuran) at, e.g., 70° C. to form Compound C′.

### Solvent and Temperature to Form Compound A

The admixing of Compound C or C′ with Compound D is conducted in a suitable solvent. As is understood, when the process is a “one-pot” process, the solvent can comprise solvent from step (a). In some embodiments, the solvent may be different than the solvent in step (a). In some embodiments, the admixing of Compound C or C′ with Compound D is conducted in a solvent comprising THF and water.

### Processes for Preparing Compound E

The disclosed processes for preparing Compound E, a stereoisomer thereof, a salt thereof, or a salt of a stereoisomer thereof, comprise admixing Compound F, or a salt thereof, with an imine reductase (IRED) to form Compound E, a stereoisomer thereof, a salt thereof, or a salt of a stereoisomer thereof. In some embodiments Compound E is, or is enriched in, the (S)-stereoisomer of Compound E. By way of example, in various embodiments, in conjunction with other above or below embodiments, (S)-Compound E produced according the disclosed processes has an enantiomeric excess of 95% or more (e.g., 95, 96, 97, 98, 99, 99.5, 99.6, 99.7, 99.8, or 99.9% or more).

In various embodiments, in conjunction with other above or below embodiments, Compound E, a stereoisomer thereof, a salt thereof, or a salt of a stereoisomer thereof, is prepared in an overall yield of 75% or more, based upon Compound F. In some embodiments, Compound E, a stereoisomer thereof, a salt thereof, or a salt of a stereoisomer thereof, is prepared in a yield of 80-90%, based upon Compound F, and with a stereochemical purity of greater than 99% ee. In some embodiments, Compound E, a stereoisomer thereof, a salt thereof, or a salt of a stereoisomer thereof, is prepared in yield of 90% or more in high stereochemical purity from a compound

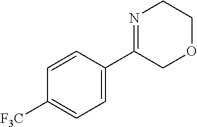 [SMILES: FC(F)(F)c1ccc(C2=NCCOC2)cc1]

(e.g., 91%, 92%, 93%, 94%, 95%, 96%, 97%, 98%, or 99% or more yield; +99% ee by chiral HPLC). In some embodiments, (S)-Compound E is prepared in 91% yield and +99% ee by chiral HPLC.

The RED enzyme can be any suitable IRED. IREDs are commercially available (e.g., Prozomix Limited (Northumberland, UK). In some embodiments, the RED used is IRED-155, sometimes alternatively referred to as IRED-0712-C.

The RED is present in a suitable amount. For example, in some embodiments, the RED is present in 5-10 wt %, based on Compound F. In some embodiments, the RED is present in an amount of 10 wt %, based on Compound F. In some embodiments, the RED is present in an amount of 5 wt % based on Compound F.

In some embodiments, the enzymatic reduction is conducted in a buffered aqueous solution. Desirably, the enzymatic reduction is conducted at a pH of 6 to 9 (e.g., a pH of 6 to 8 or 7 to 8). Suitable buffers include, for example, 2-Amino-2-(hydroxymethyl)-1,3-propanediol (Tris) and phosphate buffers. In some embodiments the buffer is a potassium phosphate buffer (pH 7.4) present in an amount of 30 volumes. In some embodiments, the buffer is potassium phosphate buffer (pH 7.4) present in an amount of 15 volumes.

The enzymatic reaction mixture comprises any suitable reductant, oxidant, and/or co-factors capable of maintaining enzymatic activity at a desired rate. By way of example, in some embodiments, the admixing of Compound F, or a salt thereof, with an RED is conducted using nicotinamide adenine dinucleotide phosphate (NADP+) (3 wt %), glucose dehydrogenase (GDH) (1.5 to 3 wt %), and glucose (reductant). In some embodiments, a slight excess of NADP+ (1.01 mmol) based upon the substrate. Similarly, an excess of the reductant can be used (e.g., 1.1 eq, 1.2 eq, 1.3 eq, 1.4 eq, or 1.5 eq reductant). In some embodiments, the enzymatic reaction mixture comprises 1.4 eq. of D-(+)-glucose.

The admixing of Compound F, or a salt thereof, with an RED is conducted at a suitable temperature. In various cases, the admixing reaction is conducted at a temperature of less than 50° C. (e.g., 45° C.). For example, in some embodiments, the admixing of Compound F, or a salt thereof, with an imine reductase is conducted at 20-45° C., 20-40° C., 20-35° C., or 30-35° C.

As described herein, in various embodiments, in conjunction with other above or below embodiments, the disclosed processes further comprise admixing Compound G, or a salt thereof, with Compound H and an organometallic reagent or magnesium metal to form Compound F′. In these embodiments, Compound F′, containing an amine protecting group, is converted to Compound F by removing the protecting group (e.g., deprotecting) from the amine group. In some embodiments, the protecting group in Compound F′ is Boc, which can be removed, e.g., using aqueous acid (e.g., HCl). An illustrative embodiment depicting the conversion of Compound F′ to Compound F and then to Compound E is depicted in Scheme 4.

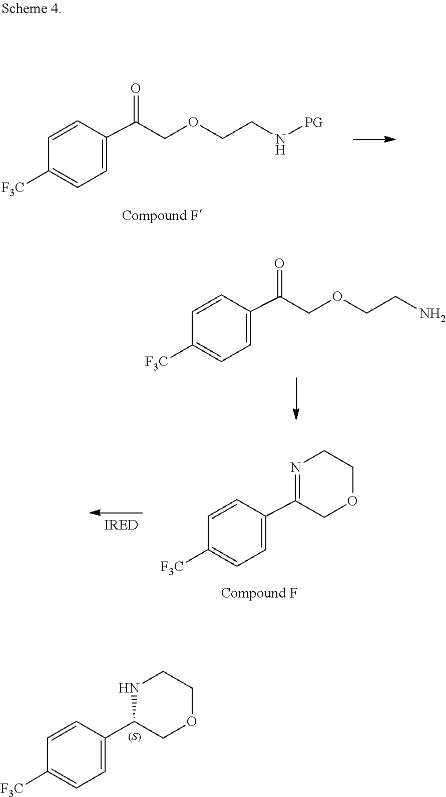

In some embodiments, Compound F, e.g.,

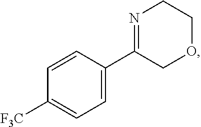 [SMILES: FC(F)(F)c1ccc(C2=NCCOC2)cc1]

is isolated prior to conversion to Compound E.

The process for preparing Compound F can be performed in either batch mode, or continuous mode.

In various cases, Compound F is prepared in a yield of 40% or more, based upon Compound G (e.g., 40, 45, 50, 55, 60, 65, 70, 75, 80, or 85% or more based upon Compound G). In some embodiments, wherein Compound G is admixed with a mixture comprising Compound H and an organometallic reagent or magnesium metal in batch mode, the yield of Compound F is 45-65%. In some embodiments, Compound F is prepared in continuous mode with a yield of 67-82%, wherein Compound G is admixed with a mixture comprising Compound H and an organometallic reagent or magnesium metal, wherein the mixture comprising Compound H and the organometallic reagent is prepared in continuous mode.

The organometallic reagent for admixing with Compound H is any suitable organometallic reagent. Nonlimiting suitable organometallic reagents include a Grignard reagent. In some embodiments, the organometallic reagent is isopropyl magnesium chloride (iPrMgCl). In some cases, an excess of the organometallic reagent is used. For example, in some embodiments 1.5 eq of iPrMgCl is used relative to Compound H. In some embodiments, Compound G is the limiting reagent, that is, less than 1 eq of Compound G (e.g., 0.95, 0.9, 0.85, or 0.8 eq), relative to Compound H, is present in the reaction. For example, in some embodiments, 0.85 eq of Compound G is added to the Grignard reagent formed from Compound H and iPrMgCl. In some embodiments, the organometallic reagent is replaced in the reaction with magnesium metal, that is, Compound G is admixed with a mixture comprising Compound H and magnesium metal to form Compound F.

### Compound I

The disclosure provides processes for preparing Compound I, a stereoisomer thereof, a salt thereof, or a salt of a stereoisomer thereof using the disclosed processes. In some embodiments, the disclosed processes comprise admixing Compound A′ (Compound A where Y¹ is —CO₂H), or a salt thereof, with Compound E, a salt thereof, or a salt of a stereoisomer thereof and a coupling agent to form Compound I, a stereoisomer thereof, a salt thereof, or a salt of a stereoisomer thereof.

The disclosed processes provide Compound I in a suitable yield. In some embodiments, Compound I is formed from the disclosed process in a chemical yield of 70% or more (e.g., 75%, 80%, 85%, or 90% or more), relative to Compound A. Moreover, the stereochemical purity of Compound I is not degraded during the reaction of Compound A1 with (S)-Compound E.

The coupling agent can be any suitable coupling agent capable of forming an amide bond between Compound A′ and Compound E as is present in Compound I. Suitable coupling agents include, for example, phosphonium and uronium salts. In some embodiments, the coupling agent is selected from the group consisting of chloro-N,N,N′,N′-tetramethylformamidinium hexafluorophosphate (TCFH), 0-[(ethoxycarbonyl)cyanomethyleneamino]-N,N,N′N′-tetramethyluronium tetrafluoroborate (TOTU), 1-cyano-2-ethoxy-2-oxoethylidenaminooxy)dimethylamino-morpholino-carbenium hexafluorophosphate (COMU), 1-[bis(dimethylamino)methylene]-1H-1,2,3-triazolo[4,5-b]pyridinium 3-oxide hexafluorophosphate (HATU), N-[(1H-benzotriazol-1-yl)-(dimethylamino)methylene]-N methylmethanaminium hexafluorophosphate N-oxide (HBTU), 0-(benzotriazol-1-yl)-N,N,N′,N′-tetramethyluronium tetrafluoroborate (TBTU), propanephosphonic acid anhydride (T3P), bis(2-oxo-3-oxazolidinyl)phosphinic chloride (BOPCl), 2-chloro-4,6-dimethoxy-1,3,5-triazine (CDMT), 1,1′-carbonyldiimidazole (CDI), and 1-cyano-2-ethoxy-2-oxoethylideneaminooxy-tris-pyrrolidino-phosphonium hexafluorophosphate (PyOxim). In some embodiments, the coupling agent is TBTU or CDI or chloro-N,N,N′,N′-tetramethylformamidinium hexafluorophosphate (TCFH). Further, in some embodiments, the coupling agent is TBTU. In some embodiments, the coupling agent is TCFH. In some embodiments, the coupling agent is CDI.

In some embodiments, in conjunction with other above or below embodiments, the admixing of Compound A′ and Compound E is performed in the presence of an additive. The presence of an additive can facilitate the coupling reaction (e.g., improved chemical yields and/or improved stereochemical purity). Suitable nonlimiting examples of additives include N-methylimidazole and alkylamine bases (e.g., trimethylamine, and diisopropylethylamine). In some embodiments, the additive is triethylamine. In some embodiments, the additive is N-methylimidazole (NMI), trimethylamine, diisopropylethylamine, or a mixture thereof. Suitable nonlimiting examples of additives include organic acids (e.g., trifluoromethane sulfonic acid, trifluoroacetic acid, acetic acid) and mineral acids (e.g., hydrochloric acid, hydrobromic acid). In some embodiments, the additive is trifluoromethane sulfonic acid. In some embodiments, the additive is hydrochloric acid.

In some embodiments, the processes for preparing Compound I, a stereoisomer thereof, a salt thereof, or a salt of a stereoisomer thereof, further comprise a purification of Compound I. For example, in some embodiments, the process further comprises crystallizing Compound I, a stereoisomer thereof, a salt thereof, or a salt of a stereoisomer thereof. In some embodiments, Compound I is recrystallized from an organic solvent comprising acetone. In some embodiments, the organic solvent further comprises an anti-solvent, such as for example a hydrocarbon solvent (e.g., heptane).

It should be appreciated that the disclosed processes for preparing Compound A and Compound E are useful for preparing Compound I. For example, in various embodiments, in conjunction with other above or below embodiments, the disclosed processes for preparing Compound I comprise preparing Compound E according to the disclosed processes herein. In some cases, where Compound A comprises a Y¹ that is not a CO₂H moiety, the processes disclosed herein can further include converting the Y¹ of Compound A to CO₂H (i.e., Compound A′). For example, if Y¹ is an ester or amide, the ester or amide is hydrolyzed to the acid. If Y¹ is an aldehyde, the aldehyde is oxidized to the acid. If Y¹ is a nitrile, the nitrile is converted to the acid. If Y¹ is a halide (e.g., chloride), the halide is converted to the acid.

In some cases, Compound I is prepared as shown in the below scheme, when Y¹ is a halide (e.g., Cl):

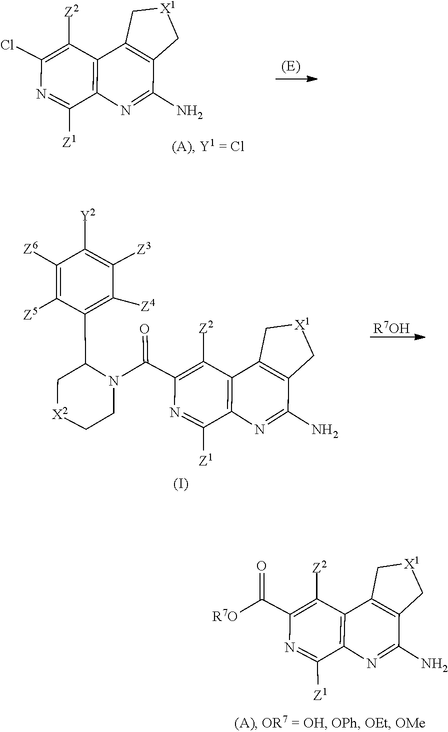

In some embodiments, Compound (A) where Y¹═Cl is admixed with metal catalyst 0.5% molar to 5.0% molar (including but not limited to palladium(II) acetate, palladium(II) chloride, [1,1′-bis(diphenylphosphino)ferrocene] dichloropalladium(II)) and ligand 0.5% molar to 10% molar (including but not limited to [1,1′-bis(diphenylphosphino)ferrocene], 1,3-bis(diphenylphosphino)propane bis(tetrafluoroborate), 1,3-bis(dicyclohexylphosphino)propane bis(tetrafluoroborate)) and carbon monoxide (20 to 100 pounds-per-square-inch; or more specifically 50 pounds-per-square-inch) and 2.0 to 10.0 molar equivalents of inorganic or organic base or mixture thereof (potassium acetate, potassium bicarbonate, potassium carbonate, 1,8-diazabicyclo[5.4.0]undec-7-ene (also known as DBU), 1,5,7-triazabicyclo[4.4.0]dec-5-ene (also known as TBD), 1,5-diazabicyclo[4.3.0]non-5-ene (also known as DBN); exact embodiment potassium carbonate and DBU), and 1.0 to 15.0 molar equivalents of nucleophile to form the desired product (nucleophile: R⁷ of product): (water: OR⁷ ═OH; ethanol: OR⁷═OCH₂CH₃; methanol: OR⁷═OCH₃; phenyl: OR⁷═OPh; Compound (E): product is Compound I) in solvent (e.g., 1-methylpyrroldine, dimethylsulfoxide, methanol, acetonitrile, acetic acid) at 85° C.

In some cases when Y¹ is halide (e.g., Cl), the halide is first converted to a CN group before Compound I is prepared:

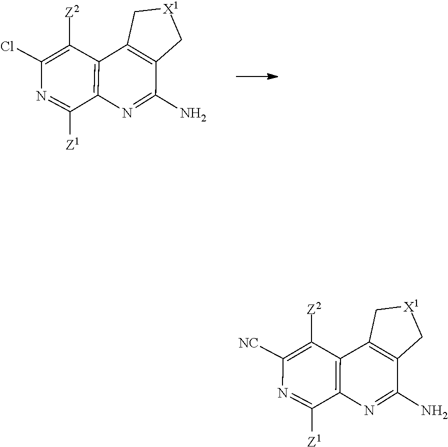

Compound A (when Y¹═Cl) is admixed with 1% to 10% molar equivalents of metal catalyst (including but not limited to bis(1,5-cyclooctadien)nickel(0) or palladium(II) acetate or palladium(II) chloride) with 1% to 10% molar equivalents of ligand (including but not limited to 4,5-bis(diphenylphosphino)9,9-dimethylxanthene, 1,1′-bis(diphenylphosphino)ferrocene, bis(2-dicyclohexylphosphinophenyl)ether) and 0.5 to 1.5 molar equivalents of zinc(II) cyanide and 1.0 to 1.5 molar equivalents of additive 4-dimethylaminopyridine and 0.1 to 1.0 molar equivalents of zinc and solvent (including but not limited to dimethylsulfoxide, N,N-dimethylacetamide) at, e.g., 80° C., to form Compound A where Y¹ is CN.

### EMBODIMENTS

1. A process for preparing Compound A, or a salt thereof:

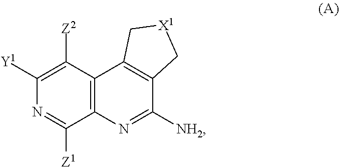

wherein
X¹ is NH, NR¹, O, S, or SO₂;
Y¹ is —CN, —Cl, —CHO, —COOH, —CONHR¹, —CON(R¹)₂, or —CO₂R¹;
each of Z¹ and Z² is independently H, F, or C₁-C₆ alkyl; and
each R¹ is independently C₁-C₆ alkyl;
   comprising
(a) admixing Compound B

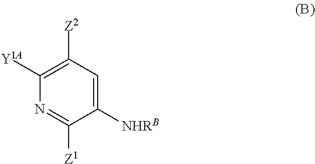

with a first transition metal catalyst and a boron-containing compound to form Compound C when R^{B} is hydrogen or to form Compound C′ when R^{B} is —COOR₄, and optionally isolating Compound C or Compound C′:

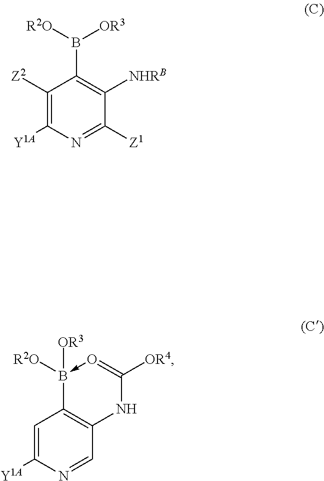

wherein R^{B} is hydrogen or —COOR⁴, each of R² and R³ is independently H or C₁-C₆ alkyl, or when taken together with the boron and oxygen atoms to which they are attached form a 5-, 6-, or 8-membered cyclic boronate; R⁴ is C₁-C₆alkyl; Y^{1A} is —CN, —Cl, —CONHR¹, —CON(R¹)₂, or —CO₂R¹; and
(b) admixing Compound C or Compound C′ with Compound D

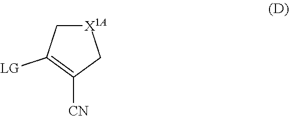

and a second transition metal catalyst to form Compound A or a salt thereof, wherein X^{1A} is NR⁷, O, or S, and R⁷ is C₁-C₆alkyl, benzyl, or p-methoxybenzyl; and LG is a leaving group.

2. The process of embodiment 1, wherein X¹ is O.

3. The process of embodiment 1 or 2, wherein Y¹ is —CN.

4. The process of embodiment 1 or 2, wherein Y¹ is —Cl.

5. The process of embodiment 1 or 2 when Y¹ of Compound A is CHO or COOH, the process further comprising converting —CN, —CONHR¹, —CON(R¹)₂ or CO₂R¹ to CHO or COOH.

6. The process of any one of embodiments 1-5, wherein X¹ of Compound A is NH, and the process further comprises converting X^{1A} to NH.

7. The process of any one of embodiments 1-6, wherein Z¹ and Z² are each H.

8. The process of any one of embodiments 1-7, wherein Compound A has a structure of A1:

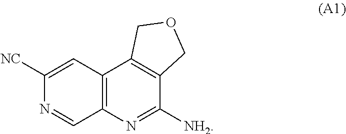 [SMILES: N#Cc1cc2c3c(c(N)nc2cn1)COC3]

9. The process of any one of embodiments 1-7, wherein Compound A has a structure of A1:

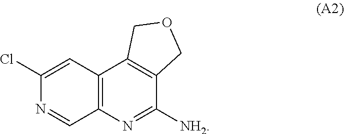 [SMILES: Nc1nc2cnc(Cl)cc2c2c1COC2]

10. The process of any one of embodiments 1-9, wherein the first transition metal catalyst comprises iridium.

11. The process of embodiment 10, wherein the first transition metal catalyst is selected from the group consisting of [Ir(OMe)(cod)]₂, [Ir(Cl)(cod)]₂.

12. The process of any one of embodiments 1-11, wherein the first transition catalyst is present in an amount of 1 to 5 mol % or wt %, based upon Compound B.

13. The process of any one of embodiments 1-12, wherein the boron-containing compound is 4,4,4′,4′,5,5,5′,5′-octamethyl-2,2′-bi(1,3,2-dioxaborolane or 4,4,5,5-tetramethyl-1,3,2-dioxaborolane.

14. The process of embodiment 13, wherein the boron-containing compound is 4,4,4′,4′,5,5,5′,5′-octamethyl-2,2′-bi(1,3,2-dioxaborolane).

15. The process of any one of embodiments 1-14, wherein Compound C has a structure of C1:

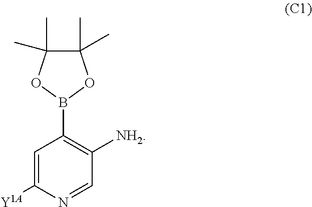

16. The process of any one of embodiments 1-14, wherein Compound C′ has a structure of C′-2:

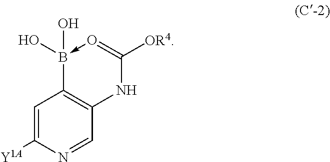

17. The process of any one of embodiments 1-14, wherein Compound C′ has a structure of C′-3

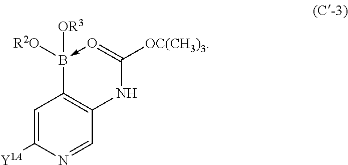

18. The process of any one of embodiments 1-14, wherein Compound C′ has a structure of C′-4, C′-5, C′6, or C′7:

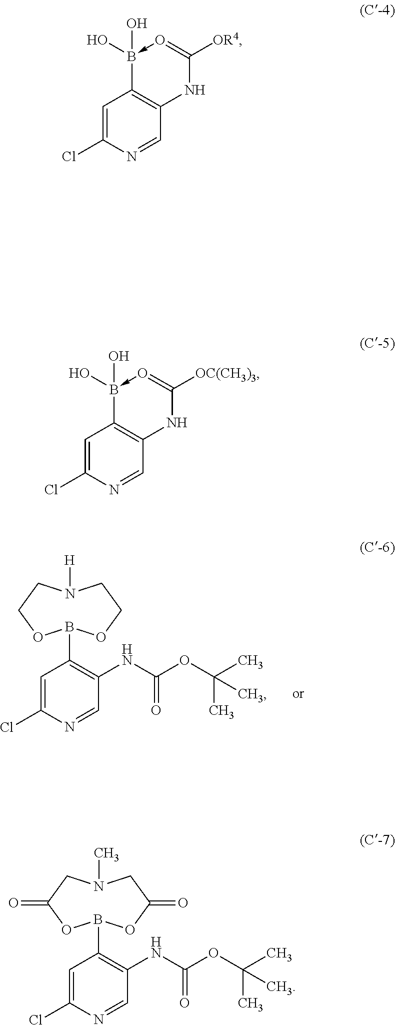

19. The process of any one of embodiments 1-18, wherein LG of Compound D is a sulfonate ester, a sulfamate, or a halide.

20. The process of embodiment 19, wherein the sulfonate ester is tosyl, mesyl, nosyl, or triflyl.

21. The process of any one of embodiments 1-20, wherein Compound D has a structure of D1:

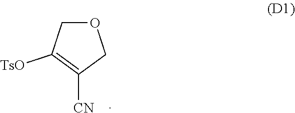 [SMILES: Cc1ccc(S(=O)(=O)OC2=C(C#N)COC2)cc1]

22. The process of any one of embodiments 1-21, wherein the second transition metal catalyst comprises a palladium catalyst, or a nickel catalyst.

23. The process of embodiment 22, wherein the second transition metal catalyst is dichloro[9,9-dimethyl-4,5-bis(diphenylphosphino)xanthene]palladium(II).

24. The process of any one of embodiments 1-23, wherein the second transition metal catalyst is present in an amount of 1 to 5 mol % or wt %, based upon Compound B.

25. The process of any one of embodiments 1-24, wherein the process is conducted in a vessel without isolating Compound C or Compound C′.

26. The process of any one of embodiments 1-25, wherein Compound C or Compound C′ is isolated.

27. The process of embodiment 26, wherein Compound C has a structure of C1-a:

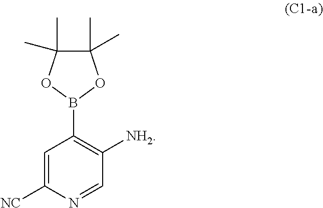 [SMILES: CC1(C)OB(c2cc(C#N)ncc2N)OC1(C)C]

28. The process of embodiment 26, wherein Compound C′ has a structure of C′-5, C′-6, or C′-7:

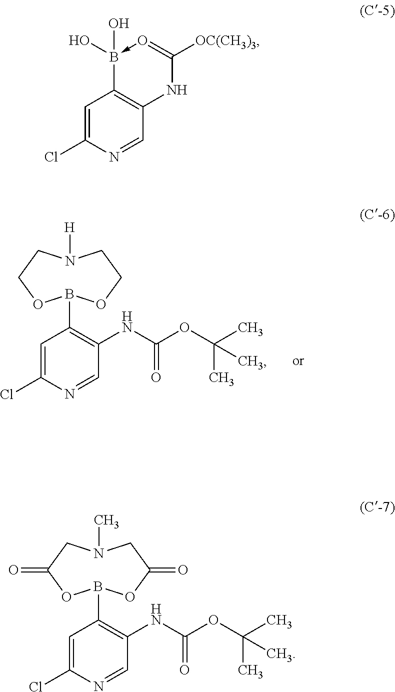

29. The process of any one of embodiments 1-28, wherein Compound A is prepared in an overall yield of 50% or more, based upon Compound B.

30. A process for preparing Compound E, a stereoisomer thereof, a salt thereof, or a salt of a stereoisomer thereof:

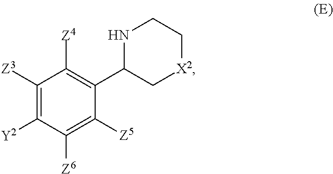

wherein
X² is NR¹, O, or S, R¹ is C₁-C₆alkyl;
Y² is H, C₁-C₆ alkyl, or C₁-C₆ haloalkyl; and
each of Z³, Z⁴, Z⁵, and Z⁶ is independently H, C₁-C₆ alkyl, or chloride; comprising
admixing Compound F, or a salt thereof, with an imine reductase (IRED) to form Compound E, a stereoisomer thereof, a salt thereof, or a salt of a stereoisomer thereof,

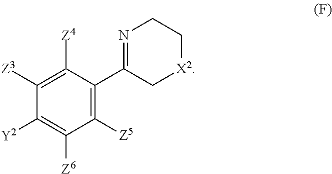

31. The process of embodiment 30, wherein X² is O.

32. The process of embodiment 30 or 31, wherein Y² is CF₃.

33. The process of any one of embodiments 30-32, wherein each of Z³, Z⁴, Z⁵, and Z⁶ is H.

34. The process of any one of embodiments 30-33, wherein Compound E is enriched in the (S)-stereoisomer:

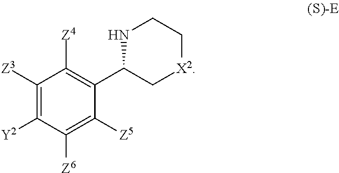

35. The process of embodiment 34, wherein Compound E has an enantiomeric excess of 95% or more.

36. The process of embodiment 35, wherein Compound E has an enantiomeric excess of 98% or more.

37. The process of embodiment 36, wherein Compound E has an enantiomeric excess of 99% or more.

38. The process of embodiment 37, wherein Compound E has an enantiomeric excess of 99.9% or more.

39. The process of any one of embodiments 30-38, wherein the admixing is conducted at a temperature of 20-50° C.

40. The process of embodiment 39, wherein the temperature is 20-35° C.

41. The process of embodiment 1, wherein the temperature is 30-35° C.

42. The process of any one of embodiments 30-41, further comprising admixing Compound G or a salt thereof, with Compound H and an organometallic reagent or magnesium metal to form Compound F′,

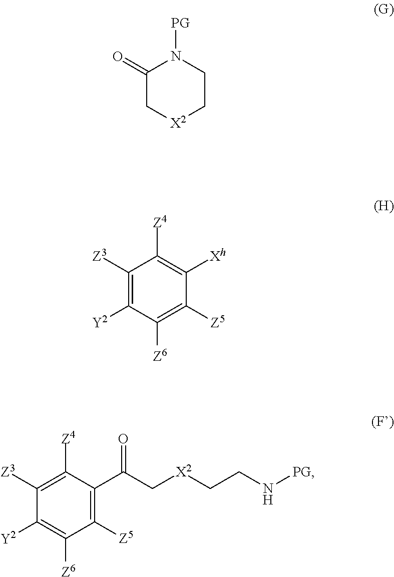

wherein PG is a protecting group and X^{h} is Cl, Br, or I.

43. The process of embodiment 42, wherein X^{h} is I.

44. The process of embodiment 42, wherein X^{h} is Br.

45. The process of embodiment 42 or 44, wherein Y² is CF₃.

46. The process of any one of embodiments 42-45, wherein each of Z³, Z⁴, Z⁵, and Z⁶ is H.

47. The process of any one of embodiments 42-446, wherein the organometallic reagent is iPrMgCl.

48. The process of any one of embodiments 42-47, wherein the protecting group is selected from the group consisting of tert-butyloxycarbonyl (Boc), benzyloxycarbonyl (Cbz), and trimethylsilyl (TMS).

49. The process of embodiment 48, wherein the protecting group is Boc.

50. The process of any one of embodiments 30-49, wherein the process is conducted in batch mode.

51. The process of any one of embodiments 30-49, wherein the process is conducted in continuous mode.

52. The process of any one of embodiments 30-51, further comprising deprotecting Compound F′ to form Compound F, or salt thereof.

53. The process of any one of embodiments 30-51, wherein Compound E, a stereoisomer thereof, a salt thereof, or a salt of a stereoisomer thereof, is prepared in an overall yield of 75% or more, based upon Compound F.

54. A process for preparing Compound I, a stereoisomer thereof, a salt thereof, or a salt of a stereoisomer thereof:

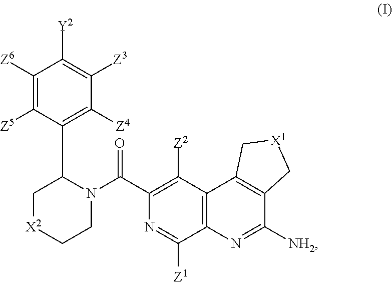

comprising
admixing Compound A′, or a salt thereof with Compound E, a stereoisomer thereof, a salt thereof, or a salt of a stereoisomer thereof, and a coupling agent to form Compound I, a stereoisomer thereof, a salt thereof, or a salt of a stereoisomer thereof,

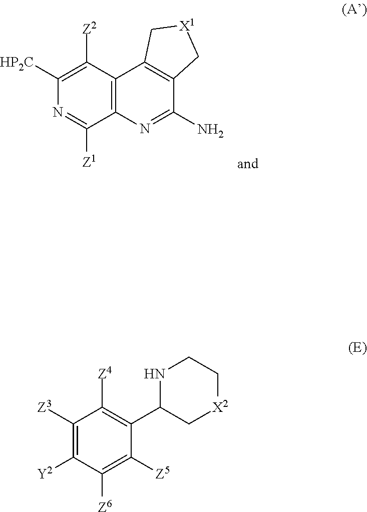

wherein
X¹ is NH, NR¹, O, S, or SO₂;
X² is NR¹, O, or S;
each R¹ is independently C₁-C₆alkyl;
Y² is H, C₁-C₆ alkyl, or C₁-C₆ haloalkyl;
each of Z¹ and Z² is independently H, F, or C₁-C₆alkyl; and
each of Z³, Z⁴, Z⁵, and Z⁶ is independently H, C₁-C₆alkyl, or chloride.

55. The process of embodiment 54, wherein Compound I is the (S)-stereoisomer:

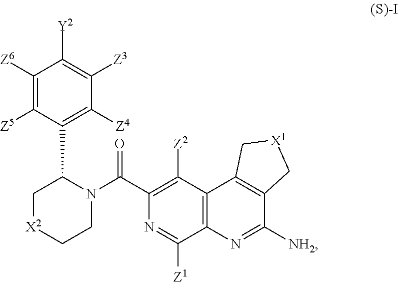

56. The process of embodiment 55, wherein X¹ and X² are each O; Y² is —CF₃; and each of Z¹, Z², Z³, Z⁴, Z⁵, and Z⁶ is H.

57. The process of any one of embodiments 54-56, wherein the coupling agent is selected from the group consisting of chloro-N,N,N′,N′-tetramethylformamidinium hexafluorophosphate (TCFH), 0-[(ethoxycarbonyl)cyanomethyleneamino]-N,N,N′N′-tetramethyluronium tetrafluoroborate (TOTU), 1-cyano-2-ethoxy-2-oxoethylidenaminooxy)dimethylamino-morpholino-carbenium hexafluorophosphate (COMU), 1-[bis(dimethylamino)methylene]-1H-1,2,3-triazolo[4,5-b]pyridinium 3-oxide hexafluorophosphate (HATU), N-[(1H-benzotriazol-1-yl)-(dimethylamino)methylene]-N methylmethanaminium hexafluorophosphate N-oxide (HBTU), 0-(benzotriazol-1-yl)-N,N,N′,N′-tetramethyluronium tetrafluoroborate (TBTU), propanephosphonic acid anhydride (T3P), bis(2-oxo-3-oxazolidinyl)phosphinic chloride (BOPCl), 2-chloro-4,6-dimethoxy-1,3,5-triazine (CDMT), 1,1′-carbonyldiimidazole (CDI), and 1-cyano-2-ethoxy-2-oxoethylideneaminooxy-tris-pyrrolidino-phosphonium hexafluorophosphate (PyOxim).

58. The process of any one of embodiments 54-57, wherein the coupling agent is O-(benzotriazol-1-yl)-N,N,N′,N′-tetramethyluronium tetrafluoroborate (TBTU).

59. The process of embodiment 57, wherein the coupling agent is CDI.

60. The process of any one of embodiments 54-59, wherein the admixing is performed in the presence of an additive.

61. The process of embodiment 60, wherein the additive is N-methylimidazole (NMI) or triethylamine.

62. The process of embodiment 60, wherein the additive is trifluoromethanesulfonic acid, hydrochloric acid, hydrobromic acid, or hydroiodic acid.

63. The process of any one of embodiments 54-62, further comprising crystallizing Compound I, a stereoisomer thereof, a salt thereof, or a salt of a stereoisomer thereof.

64. The process of any one of embodiments 54-63, wherein Compound E is prepared according to the process of any one of embodiments 30-53.

65. The process of any one of embodiments 54-64, the process further comprising converting Compound A to Compound A′, and Compound A is prepared by the process of any one of embodiments 1-29.

66. The process of any one of embodiments 54-65, wherein Compound I is (4-amino-1,3-dihydrofuro[3,4-c][1,7]naphthyridin-8-yl)-[(3S)-3-[4-(trifluoromethyl)phenyl]morpholin-4-yl]methanone.

67. The process of embodiment 66, wherein Compound I is enriched in the (S)-stereoisomer.

68. The process of embodiment 66, wherein Compound I has the structure:

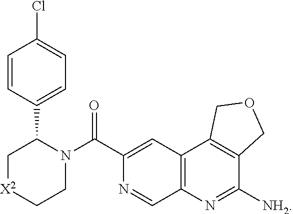

69. The process of any one of embodiments 1-28 or 65-67, wherein Compound B has a structure of B1

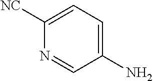

### (Compound B1) or B1′

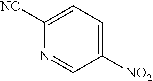 [SMILES: N#Cc1ccc([N+](=O)[O-])cn1]

### (Compound B1′).

70. The process of embodiment 69, further comprising admixing 2-cyano-5-nitropyridine

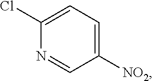

or 2-chloro-5-nitropyridine

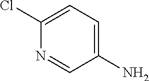

or a salt thereof with a nitroreductase in a solvent to form Compound B1, Compound B1′, or a salt thereof.

71. The process of embodiment 70, wherein the nitroreductase is selected from the group consisting of NR-17, NR-X4-mut2, NR-X4-mut10, NR-X18, NR-X27, NR-X30, NR-X32, NR-X36, NR-X39, NR-X41, NR-X53, NR-X54 and a combination thereof.

72. The process of embodiment 70 or 71, wherein the nitroreductase is NR-17 or NR-X36.

73. The process of embodiment 72, wherein NR-17 or NR-X36 is present in an amount of 0.1-10 wt %, based upon 2-cyano-5-nitropyridine or 2-chloro-5-nitropyridine.

74. The process of embodiment 73, wherein NR-17 or NR-X36 is present in an amount of 5-7 wt %, based upon 2-cyano-5-nitropyridine or 2-chloro-5-nitropyridine.

75. The process of any one of embodiments 70-74, further comprising admixing 2-cyano-5-nitropyridine, 2-chloro-5-nitropyridine, or a salt thereof and the nitroreductase in the presence of one or more of a glucose dehydrogenase (GDH), a third transition metal catalyst, a co-factor, a reductant, or a buffer.

76. The process of embodiment 75, wherein the third transition metal catalyst comprises vanadium, iron, copper, or a combination thereof.

77. The process of embodiment 75 or 76, wherein the vanadium is a vanadium oxide.

78. The process of any one of embodiments 75-77, wherein the vanadium oxide is a vanadium(IV) oxide or a vanadium(V) oxide.

79. The process of any one of embodiments 75-78, wherein the third transition metal catalyst is ammonium metavanadate (NH₄VO₃) or vanadium pentoxide (V₂O₅).

80. The process of any one of embodiments 73-77, wherein the third transition metal catalyst is present in an amount of 0.01-2.5 eq, based upon 2-cyano-5-nitropyridine or 2-chloro-5-nitropyridine.

81. The process of embodiment 80, wherein the third transition metal catalyst is present in an amount of 0.1 eq, based upon 2-cyano-5-nitropyridine or 2-chloro-5-nitropyridine.

82. The process of embodiment 80, wherein the third transition metal catalyst is present in an amount of 2 eq, based upon 2-cyano-5-nitropyridine or 2-chloro-5-nitropyridine.

83. The process of any one of embodiments 75-82, wherein the glucose dehydrogenase is selected from the group consisting of GDH-101, GDH-105, CDX-901, and a combination thereof.

84. The process of any one of embodiments 75-83, wherein the glucose dehydrogenase is GDH-101.

85. The process of any one of embodiments 75-84, wherein the glucose dehydrogenase is present in an amount of 0.1-25 wt %, based upon 2-cyano-5-nitropyridine or 2-chloro-5-nitropyridine.

86. The process of embodiment 85, wherein the glucose dehydrogenase is present in an amount of 1 wt %, based upon 2-cyano-5-nitropyridine or 2-chloro-5-nitropyridine.

87. The process of any one embodiments 75-86, wherein the co-factor is selected from the group consisting of nicotinamide adenine dinucleotide (NAD+), dihydronicotinamide adenine dinucleotide (NADH), nicotinamide adenine dinucleotide phosphate (NADP+), dihydronicotinamide adenine dinucleotide phosphate (NADPH), a salt of NADPH, and a combination thereof.

88. The process of any one of embodiments 75-87, wherein the co-factor is nicotinamide adenine dinucleotide phosphate (NADP+).

89. The process of any one of embodiments 75-88, wherein the co-factor is present in an amount of 0.5-20 wt %, based upon 2-cyano-5-nitropyridine or 2-chloro-5-nitropyridine.

90. The process of embodiment 89, wherein the co-factor is present in an amount of 0.7 wt %, based upon 2-cyano-5-nitropyridine.

91. The process of any one of embodiments 75-90, wherein the reductant is glucose.

92. The process of any one embodiments 75-91, wherein the reductant is present in an amount of 3-5 eq, based upon 2-cyano-5-nitropyridine or 2-chloro-5-nitropyridine.

93. The process of embodiment 92, wherein the reductant is present in an amount of 3.1 eq, based upon 2-cyano-5-nitropyridine or 2-chloro-5-nitropyridine.

94. The process of any one of embodiments 75-93, wherein the buffer comprises a tricine buffer, a potassium phosphate buffer, 4-(2-hydroxyethyl)-1-piperazineethanesulfonic acid (HEPES), tris(hydroxymethyl)aminomethane (Tris), or a combination thereof.

95. The process of any one of embodiments 75-94, wherein the buffer is a potassium phosphate buffer.

96. The process of any one of embodiments 75-95, wherein the buffer maintains a pH of 6 to 9.

97. The process of embodiment 96, wherein the buffer maintains a pH of 7.2 to 7.5.

98. The process of any one of embodiments 75-97, wherein the buffer is present in an amount of 100-250 mM.

99. The process of embodiment 98, wherein the buffer is present in an amount of 80-95% (v/w).

100. The process of embodiment 99, wherein the buffer is present in an amount of 92% (v/w).

101. The process of any one of embodiments 70-100, wherein the admixing of 2-cyano-5-nitropyridine, 2-chloro-5-nitropyridine, or salt thereof with the nitroreductase is conducted in a solvent comprising water, dimethylsulfoxide (DMSO), toluene, methyl tert-butyl ether (MTBE), isopropyl acetate, or a combination thereof.

102. The process of any one of embodiments 70-101, wherein the solvent comprises DMSO.

103. The process of embodiment 102, wherein the solvent comprises 0.5-20 volumes of DMSO, based upon 2-cyano-5-nitropyridine.

104. The process of embodiment 103, wherein the solvent comprises 0.5 volumes of DMSO, based upon 2-cyano-5-nitropyridine.

105. The process of any one of embodiments 70-104, wherein the admixing of 2-cyano-5-nitropyridine, 2-chloro-5-nitropyridine, or salt thereof with the nitroreductase is conducted at a temperature of 20-50° C.

106. The process of embodiment 105, wherein the temperature is 32-38° C.

107. The process of any one embodiments 70-106, wherein the admixing of 2-cyano-5-nitropyridine, 2-chloro-5-nitropyridine, or salt thereof with the nitroreductase is conducted in a fed-batch mode.

### EXAMPLES

The following examples further illustrate the disclosed processes, but of course, should not be construed as in any way limiting their scope.

The following abbreviations are used herein: NMR refers to nuclear magnetic resonance; SFC refers to supercritical fluid chromatography; DIPEA refers to diisopropylethylamine; DMF refers to dimethylfomamide; PyBroP refers to benzotriazol-1-yloxytripyrrolidinophosphonium hexafluorophosphate; NaHCO₃ refers to sodium bicarbonate; EtOAc refers to ethyl acetate; EtOH refers to ethanol; DCM refers to dichloromethane; TEA refers to trimethylamine; ESI refers to electrospray ionization; DMSO refers to dimethylsulfoxide; nd refers to not detected; V or vol refers to volume (L/kg), and GC refers to gas chromatography.

### Comparative Process Example

### Comparative Example 1—(4-amino-1,3-dihydrofuro[3,4-c][1,7]naphthyridin-8-yl)(2-(4-(trifluoromethyl)phenyl)piperidin-1-yl)methanone

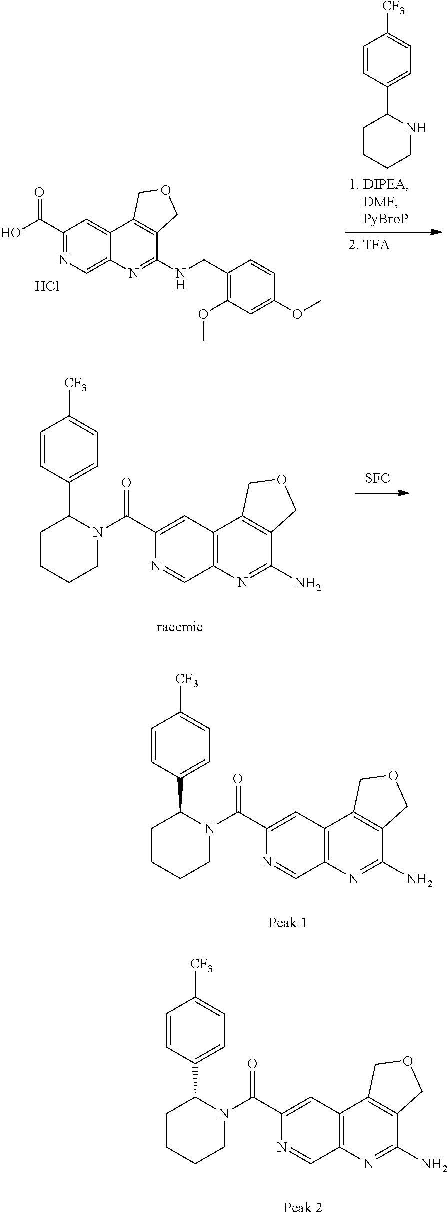

To a solution of 2-(4-(trifluoromethyl)phenyl)piperidine (0.100 g, 0.436 mmol, Arch Corporations, NJ), 4-((2,4-dimethoxybenzyl)amino)-1,3-dihydrofuro[3,4-c][1,7]naphthyridine-8-carboxylic acid hydrochloride (0.273 g, 0.654 mmol) and 1,1′-dimethyltriethylamine (0.564 g, 0.762 mL, 4.36 mmol, Sigma-Aldrich Corporation) in DMF (4 mL) was added bromotripyrrolidinophosphonium hexafluorophosphate (0.203 g, 0.436 mmol, Sigma-Aldrich Corporation) and the resulting mixture was heated at 50° C. for 30 min. The reaction was brought to rt, diluted with water, sat. NaHCO₃ and extracted with EtOAc (3×). The combined organics were dried over Na₂SO₄, filtered and concentrated. The residue was then chromatographed on silica gel using 0-50% (3:1 EtOAc/EtOH) in heptane to afford (4-((2,4-dimethoxybenzyl) amino)-1,3-dihydrofuro[3,4-c][1,7]naphthyridin-8-yl)(2-(4-(trifluoromethyl)phenyl)piperidin-1-yl)methanone as a light yellow solid. m/z (ESI): 593 (M+H)⁺.

To a solution of (4-((2,4-dimethoxybenzyl)amino)-1,3-dihydrofuro[3,4-c][1,7]naphthyridin-8-yl)(2-(4-(trifluoromethyl)phenyl)piperidin-1-yl)methanone in DCM (2 mL) was added TFA (14.80 g, 10 mL, 130 mmol, Aldrich) and the resulting mixture was heated at 50° C. for 1 h. The reaction was concentrated, washed with 10% Na₂CO₃ and extracted with DCM. The combined organics were concentrated and chromatographed on silica gel using 0-50% (3:1 EtOAc/EtOH) to afford (4-amino-1,3-dihydrofuro[3,4-c][1,7]naphthyridin-8-yl)(2-(4-(trifluoromethyl)phenyl)piperidin-1-yl)methanone (0.042 g, 0.095 mmol, 21.76% yield) as an off-white solid. m/z (ESI): 443 (M+H)⁺.

The compound was purified via preparative SFC using a Chiral Technologies AS column (250×21 mm, 5 mm) with a mobile phase of 75% liquid CO₂ and 25% MeOH with 0.2% TEA using a flowrate of 80 mL/min. to generate 13.5 mg of peak 1 with an ee of >99% and 13 mg of peak 2 with an ee of >99% with stereochemistry arbitrarily assigned. Peak 1: (S)-(4-amino-1,3-dihydrofuro[3,4-c][1,7]naphthyridin-8-yl)(2-(4-(trifluoromethyl)phenyl)piperidin-1-yl)methanone (0.013 g, 0.029 mmol). White solid. m/z (ESI): 443 (M+H)⁺. ¹H NMR (400 MHz, DMSO-d₆) δ ppm 8.69-8.99 (m, 1H), 7.73-7.86 (m, 3H), 7.57-7.67 (m, 2H), 7.03 (br s, 2H), 5.38 (br s, 2H), 5.05 (br s, 2H), 3.64-3.91 (m, 1H), 2.35-2.46 (m, 2H), 1.86-2.01 (m, 1H), 1.29-1.72 (m, 5H). Peak 2: 3415634 #1 (R)-(4-amino-1,3-dihydrofuro[3,4-c][1,7]naphthyridin-8-yl)(2-(4-(trifluoromethyl) phenyl) piperidin-1-yl)methanone (0.011 g, 0.025 mmol). White solid. 126773-15-2 m/z (ESI): 443 (M+H)⁺. ¹H NMR (400 MHz, DMSO-d₆) δ ppm 8.81-8.98 (m, 1H), 7.74-7.84 (m, 3H), 7.62 (br d, J=7.9 Hz, 2H), 7.03 (br s, 2H), 5.39 (br d, J=2.9 Hz, 2H), 5.05 (br s, 2H), 3.72-3.87 (m, 1H), 2.36-2.45 (m, 2H), 1.85-2.04 (m, 1H), 1.31-1.72 (m, 5H).

(4-amino-1,3-dihydrofuro[3,4-c][1,7]naphthyridin-8-yl)-[3-[4-(trifluoromethyl)phenyl]morpholin-4-yl]methanone was prepared in a similar fashion as above. The enantiomers were separated as outlined in Table 1.

TABLE 1

| | | m/z | |
|---|---|---|---|
| | | (ESI): | |
| Structure | Name | (M + H)⁺ | SFC Conditions |
| 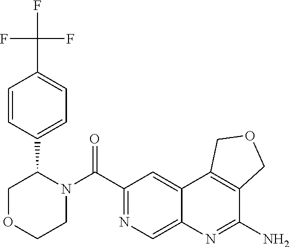 [SMILES: Nc1nc2cnc(C(=O)N3CCOC[C@@H]3c3ccc(C(F)(F)F)cc3)cc2c2c1COC2] | (4-amino-1,3- dihydrofuro[3,4- c][1,7]naphthyridin-8-yl)- [(3S)-3-[4- (trifluoromethyl)phenyl] morpholin-4-yl]methanone | 445.0 | Peak 1/SFC using a Chiral Technologies AD column (150 × 21 mm, 5 mm) with a mobile phase of 60% liquid CO₂ and 40% MeOH with 0.2% TEA using a flowrate of 80 mL/min. |
| 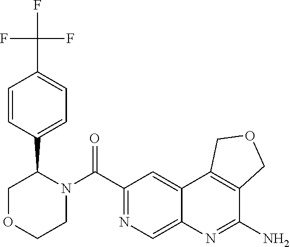 [SMILES: Nc1nc2cnc(C(=O)N3CCOC[C@H]3c3ccc(C(F)(F)F)cc3)cc2c2c1COC2] | (4-amino-1,3- dihydrofuro[3,4- c][1,7]naphthyridin-8-yl)- [(3R)-3-[4- (trifluoromethyl)phenyl] morpholin-4-yl]methanone | 445.0 | Peak 2/SFC using a Chiral Technologies AD column (150 × 21 mm, 5 mm) with a mobile phase of 60% Liquid CO₂ and 40% MeOH with 0.2% TEA using a flowrate of 80 mL/min. |

### Example 1. Synthesis of Compound A1—4-amino-1,3-dihydrofuro[3,4-c][1,7]naphthyridine-8-carbonitrile

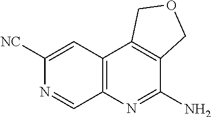 [SMILES: N#Cc1cc2c3c(c(N)nc2cn1)COC3]

### Potassium 4-cyano-2,5-dihydrofuran-3-olate

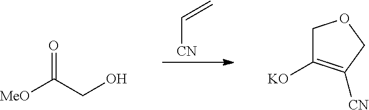

### Synthesis 1

To a solution of potassium tert-butoxide (124.5 g, 1.1 mol, 1.0 eq) in tetrahydrofuran (3.0 L, 30 V), was added a solution of methyl 2-hydroxyacetate (100 g, 1.1 mol, 1.0 eq) in tetrahydrofuran (500 mL, 5.0 V) at 0-10° C. using addition funnel over 30-45 min. Other suitable bases include potassium carbonate, sodium carbonate, and sodium bicarbonate. The resulting solution was stirred for another 15-20 min at 0-10° C. A solution of acrylonitrile (88.3 g, 1.7 mol, 1.5 eq) in tetrahydrofuran (1.0 L, 10 V) was then added slowly to the above reaction mass over a period of 3.5-4 h at 5-10° C. Other suitable solvents include MTBE, After stirring for 1 h at 5-10° C., the reaction mixture was quenched with water (20 mL, 1.1 mol, 1.0 eq), stirred for 30 min at 5-10° C. and the resulting slurry was filtered and the obtained solid was washed with THE (200 mL, 2.0 V) to get the desired product potassium 4-cyano-2,5-dihydrofuran-3-olate. Analytical data: ¹H NMR (400 MHz, DMSO-d₆): 4.51 (t, J=2.0 Hz, 2H), 3.70 (t, J=2.0 Hz, 2H).

### Synthesis 2

To a solution of potassium tert-butoxide (18.7 g, 167 mmol, 1.0 eq) in 2-methyltetrahydrofuran (450 mL, 30 L/kg), was added a solution of methyl 2-hydroxyacetate (15.0 g, 167 mmol, 1.0 eq) in 2-methyltetrahydrofuran (75.0 mL, 5.0 L/kg) at 0-10° C. over 30 min. A solution of acrylonitrile (19.4 g, 366 mmol, 2.2 equiv) in tetrahydrofuran (150 mL, 10 L/kg) was added slowly over 4 h at 5-10° C. After stirring for 1 h at 5-10° C., the reaction mixture was quenched with water (3.0 mL, 167 mmol, 1.0 eq), then the slurry was stirred for 30 min at 5-10° C., filtered and washed with 2-MeTHF (30 mL, 2.0 L/kg) to produce the desired product potassium 4-cyano-2,5-dihydrofuran-3-olate. Analytical data: ¹H NMR (400 MHz, DMSO-d₆): 4.51 (t, J=2.0 Hz, 2H), 3.70 (t, J=2.0 Hz, 2H).

### Compound D—(4-Cyano-2,5-dihydrofuran-3-yl) 4-methylbenzenesulfonate

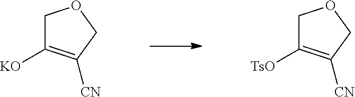

### Synthesis 1

To a slurry of potassium 4-cyano-2,5-dihydrofuran-3-olate (3.0 g, 20.1 mmol, 1.0 eq, 88.0% w/w) in 2-MeTHF (30 mL, 10.0 V), was added potassium carbonate (2.8 g, 20 mmol, 1 eq) and tosyl chloride (3.9 g, 20 mmol, 1 eq) sequentially at 20-25° C. and the resulting slurry was stirred for 2-3 h at 20-25° C. The reaction was monitored by gas chromatography (GC). The reaction mixture was filtered and the filtrate was washed with 1.5 N aqueous HCl (5 V) solution followed by 10% aqueous sodium bicarbonate solution (5 V). The organic phase was separated and concentrated under reduced pressure to give the product. Analytical data: ¹H NMR (400 MHz, CDCl₃): 2.50 (s, 3H), 4.72 (t, J=4.8 Hz, 2H), 4.85 (t, J=4.8 Hz, 2H), 7.46 (d, J=8.4 Hz, 1H), 7.90 (d, J=8.4 Hz, 1H).

### Synthesis 2

To a solution of potassium 4-cyano-2,5-dihydrofuran-3-olate (4.6 g active, 31.5 mmol; 6.6 g total mass) in acetonitrile (35 mL, 7.6 L/kg), was added potassium carbonate (8.0 g, 58 mmol, 1.8 equiv), p-toluenesulfonyl chloride (9.3 g, 49 mmol, 1.5 equiv), and 4-dimethylaminopyridine (770 mg, 6.3 mmol, 0.20 equiv) at 20° C. Other suitable bases include amine bases, for example, diisopropylethylamine, diisopropylamine, pyridine, 2,6-lutidine, 2,4,6-collidine, as well as carbonates such as sodium carbonate, sodium bicarbonate. The reaction mixture was stirred for 2-3 h at 20° C. The reaction mixture was filtered and the solids were washed with MeCN (10 mL, 2.2 L/kg). Other suitable solvents include tetrahydrofuran, 2-methyltetrahydrofuran, methyl tert-butyl ether, and isopropyl acetate. The MeCN solution was added dropwise to a stirring sample of water (96 mL, 21 L/kg), then the mixture was stirred for 1 h. The product was filtered and washed with water (15 mL, 3 L/kg). The product was dried under nitrogen at ambient temperature to produce (4-cyano-2,5-dihydrofuran-3-yl) 4-methylbenzenesulfonate. Analytical data: ¹H NMR (400 MHz, CDCl₃): 2.50 (s, 3H), 4.72 (t, J=4.8 Hz, 2H), 4.85 (t, J=4.8 Hz, 2H), 7.46 (d, J=8.4 Hz, 1H), 7.90 (d, J=8.4 Hz, 1H).

### Synthesis 3

To a solution of potassium tert-butoxide (12.4 g, 167 mmol, 1.0 equiv) in 2-methyltetrahydrofuran (300 mL, 30 L/kg), was added a solution of methyl 2-hydroxyacetate (10.0 g, 111 mmol, 1.0 equiv) in 2-methyltetrahydrofuran (50.0 mL, 5.0 L/kg) at 0-10° C. over 30 min. A solution of acrylonitrile (12.7 g, 244 mmol, 2.2 equiv) in 2-methyltetrahydrofuran (100 mL, 10 L/kg) was added slowly over 4 h at 5-10° C. After stirring for 1 h at 5-10° C., the reaction mixture was filtered and washed with 2-MeTHF (30 mL, 2.0 L/kg). To the resulting solution was added p-toluenesulfonyl chloride (21.2 g, 111 mmol, 1.0 equiv) and 4-dimethylaminopyridine (2.7 g, 22.2 mmol, 0.20 equiv). After stirring at 20° C. for 18 h, the reaction mixture was quenched with 10% w/w aqueous sodium bicarbonate (50 mL, 5.0 L/kg) and the layers were separated. The organics were then washed with water (20 mL, 2.0 L/kg) and the layers were separated. The combined organics were distilled to a total volume of 30 mL to remove water, then heptane (80 mL, 8.0 L/kg) was added slowly. The product was filtered and washed with 10% 2-MeTHF/heptane (20 mL, 2.0 L/kg). The cake was dried at ambient temperature under nitrogen to produce (4-cyano-2,5-dihydrofuran-3-yl) 4-methylbenzenesulfonate. Analytical data: ¹H NMR (400 MHz, CDCl₃): 2.50 (s, 3H), 4.72 (t, J=4.8 Hz, 2H), 4.85 (t, J=4.8 Hz, 2H), 7.46 (d, J=8.4 Hz, 1H), 7.90 (d, J=8.4 Hz, 1H).

### Compound A1—4-Amino-1,3-dihydrofuro[3,4-c][1,7]naphthyridine-8-carbonitrile

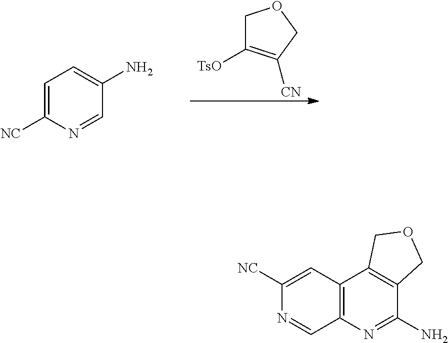 [SMILES: N#Cc1ccc(N)cn1 ; Cc1ccc(S(=O)(=O)OC2=C(C#N)COC2)cc1 ; N#Cc1cc2c3c(c(N)nc2cn1)COC3]

To a solution of 5-amino-2-pyridinecarbonitrile (800 g, 6.7 mol, 1 eq) in THE (6.4 L, 8 V) in a 30 L jacketed glass reactor at 40-45° C., 4,4,5,5 tetramethyl-1,3,2-dioxaborolane (1.3 kg, 9.9 mol, 1.5 eq) was added under nitrogen atmosphere over a period of 25 min while maintaining an internal temperature of less than about 50° C. The reaction mixture was heated to 50° C. for 1 h and then cooled back to 20-25° C. To the cooled solution was added a solution of bis(pinacolato)diboron (854 g, 3.3 mol, 0.5 eq), 4,4′-di-tert-butyl-2-2′-dipyridyl (54.3 g, 0.2 mol, 0.03 eq), [Ir(OMe)(cod)]₂ (67 g, 0.10 mol, 0.015 eq) in THE (3.2 L, 4 V) over a period of 20 min while maintaining the temperature between 25-35° C. The reaction was heated to 60-65° C. for 2-3 h, then cooled to 40-45° C., and quenched by addition of isopropyl alcohol (800 mL, 1V) over a period of 30 min at 40-45° C. and further stirred for 20 min at same temperature. The reaction was cooled to 20-25° C. and purged with nitrogen gas for 1 h. A degassed solution of K₃PO₄ (4.7 kg, 20.1 mol, 3 eq) in water (8 L, 10 V) followed by PdCl₂(Xantphos) (250 g, 3.3 mol, 0.05 eq) and (4-cyano-2,5-dihydrofuran-3-yl) 4-methylbenzenesulfonate (1782 g, 6.72 mol, 1 eq) were added under nitrogen atmosphere at 25-30° C. The reaction was heated to 60-65° C. for 2 h. The reaction completion was confirmed by HPLC and the reaction was cooled back to 20-25° C. Acetonitrile (4 L, 5V) was slowly added, stirred for 2-3 h and the slurry was filtered through Buchner funnel. The obtained cake was washed with water (8 L, 10 V) and then with dimethylacetamide (DMAc) (4 L, 5 V) and dried under vacuum for 4-5 h. The crude material and DMAc (9.6 L, 12 V) were transferred to the 30 L glass reactor followed by the addition of 1,2-bis(diphenylphosphino)ethane (136 g, 0.341 mol, 0.05 eq) at 20-25° C. and the resulting mixture was heated to 60-65° C. for 5-6 h. The reaction mass was cooled back to 20-25° C., stirred at same temperature for 1 h, filtered and the obtained solid was washed with DMAc (9.6 L, 12 V), water (8 L, 10 V) and n-heptane (2.5 L, 3 V) and dried to yield 977 g of product. The isolated material (977 g) and IPA (9.5 L, 12 V) were added to the 30 L reactor and heated to 55-60° C. for 2 h, cooled to 20-30° C. and stirred for 30 min and filtered and dried to yield the product. ¹H NMR (400 MHz, TFA-d): 9.40 (s, 1H), 8.30 (s, 1H), 5.76 (d, J=3.2 Hz, 2H), 5.59 (s, 2H). LCMS: 213.1 (M+H)⁺.

### Example 2. Synthesis of Compound A′—4-Amino-1,3-dihydrofuro[3,4-c][1,7]naphthyridine-8-carboxylic acid

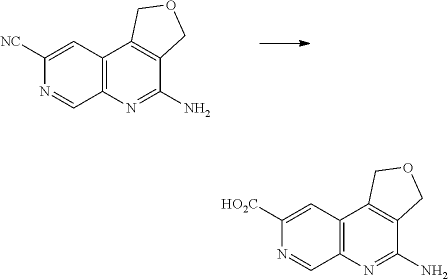 [SMILES: N#Cc1cc2c3c(c(N)nc2cn1)COC3 ; Nc1nc2cnc(C(=O)O)cc2c2c1COC2]

4-Amino-1,3-dihydrofuro[3,4-c][1,7]naphthyridine-8-carbonitrile (2.0 kg, 1.0 equiv) was charged to a clean, dry, 100 L, jacketed reactor followed by water (20.0 L). NaOH (10 N, 4.2 equiv, 4.0 L) was charged followed by an additional amount of water (16.1 L). The mixture was heated to 85±5° C. and stirred for ≥17 hours. The mixture was then cooled to 20±5° C. and drained to carboys. The reactor was rinsed with water and the process stream is polish-filtered back into the reactor. After heating the reaction to 55±5° C., HCl (37 wt %, 2.2 equiv, 1.7 L) was added while keeping the temperature less than about 60° C. Additional HCl (37 wt %, 3.0 equiv, 2.3 L) was added to the mixture over about 2 hours while keeping the temperature less than about 60° C. The product slurry was aged at 55±5° C. for about 0.5 hours, cooled to 20±5° C. over about 2 hours then aged for an additional 1.0 hour. The product slurry was filtered and the cake was washed twice with water (2×4.0 L) then twice with isopropanol (2×4.0 L). The product cake was then dried under vacuum and nitrogen stream to afford the product. LCMS: 232.08 (M+H)⁺. ¹H NMR (400 MHz, 1:1 TFA-d/Toluene-d8): 9.30 (s, 1H), 8.29 (s, 1H), 5.11 (app s, 4H).

### Example 3. Synthesis of (S)-Compound E—(3S)-3-[4-(trifluoromethyl)phenyl]morpholine

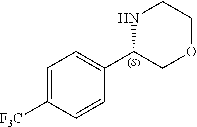

### Compound F′—tert-butyl (2-(2-oxo-2-(4-(trifluoromethyl)phenyl)ethoxy)ethyl)carbamate

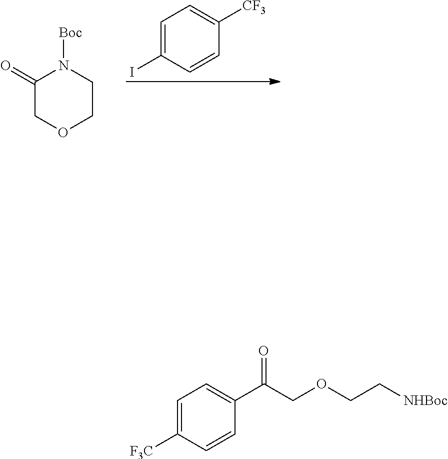

To a solution of 1-iodo-4-(trifluoromethyl)benzene (5.0 g, 18.4 mmol, 1.0 equiv) (Compound H) in toluene (20 mL, 4 V) cooled to 0° C. was added a 2 M THE solution of isopropylmagnesium chloride (13.8 mL, 27.6 mmol, 1.5 equiv). The solution was stirred 2 hrs before being cooled to −20° C. Next, a solution of tert-butyl 3-oxomorpholine-4-carboxylate (3.2 g, 15.6 mmol, 0.85 equiv) (Compound G) in toluene (15 mL, 3 V) was added slowly and stirred at −20° C. for two hours. The reaction was quenched and worked up and purified by slurring twice in DCM/Heptane (1:40) to afford the title compound tert-butyl (2-(2-oxo-2-(4-(trifluoromethyl)phenyl)ethoxy)ethyl)carbamate (Compound F′). LCMS: 248 (M+H-Boc)⁺.

### 5-(4-(trifluoromethyl)phenyl)-3,6-dihydro-2H-1,4-oxazine

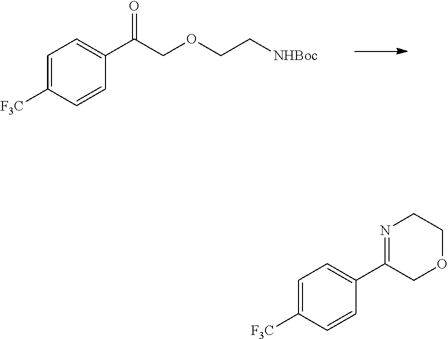 [SMILES: CC(C)(C)OC(=O)NCCOCC(=O)c1ccc(C(F)(F)F)cc1 ; FC(F)(F)c1ccc(C2=NCCOC2)cc1]

### Synthesis 1

To a solution of aqueous 2M hydrochloric acid (137 mL, 274 mmol, 2.5 equiv) was added tert-butyl (2-(2-oxo-2-(4-(trifluoromethyl)phenyl)ethoxy)ethyl)carbamate (38.2 g, 111.3 mmol, 1.0 equiv) (Compound F′) at room temperature. The resulting reaction mass was stirred and heated to 50° C. over a period of 2.5 to 3.5 hours until complete deprotection of the Boc-group was observed by HPLC analysis. The reaction was cooled to room temperature and polished filtered. In a separate vessel, potassium carbonate (36.89 g, 266.9 mmol, 2.4 equiv) was added to water (380 mL, 10 V) and stirred until a clear solution. The solution of the intermediate 2-(2-aminoethoxy)-1-(4-(trifluoromethyl)phenyl)ethan-1-one in aqueous hydrochloric acid was slowly added to the solution of potassium carbonate in water over a period ≥15 minutes. Following the addition, the reaction slurry was stirred 5-10 minutes and then filtered. The cake was washed with water (190 mL, 5 V) and immediately dried under vacuum with nitrogen purge to afford 5-(4-(trifluoromethyl)phenyl)-3,6-dihydro-2H-1,4-oxazine. LCMS: 248.02 (M+H+H₂O)⁺. ¹H NMR (400 MHz, CDCl₃): 7.81 (d, J=8.29 Hz, 2H), 7.67 (d, J=8.29 Hz, 2H), 4.65 (t, J=2.49 Hz, 2H), 3.93 (m, 2H), 3.80 (m, 2H).

### Synthesis 2

To a reactor were charged dimethylsulfoxide (600 mL, 3 L/kg) and tert-butyl (2-(2-oxo-2-(4-(trifluoromethyl)phenyl)ethoxy)ethyl)carbamate (200 g, 576 mmol). Other suitable solvents include, for example, polar aprotic solvents, including N-methyl pyrrolidinone, N,N-dimethylacetamide, or 1,3-dimethyl-2-imidazolidinone. The mixture was heated to 40° C. to dissolve the batch. To the resulting solution was slowly added 1N hydrochloric acid (2.59 L, 4.5 equiv). Other suitable mineral acids include phosphoric acid, sulfuric acid; and also organic acids including trifluoroacetic acid. The reaction mixture was heated to 60° C. for 2.5 hours, then cooled to 20° C. and polish-filtered. The reaction mixture was added slowly to a sparged pre-mixed solution of sodium carbonate (183 g, 3.0 equiv) in water (2.0 L, 10 L/kg). Other suitable inorganic bases include sodium hydroxide, and potassium carbonate. After stirring at 20° C. for 30 min, the batch was filtered. The solids were washed with 10% DMSO/water (600 mL, 3 L/kg), then washed twice with water (600 mL, 3 L/kg). The cake was dried at ambient temperature under a nitrogen stream to provide 5-(4-(trifluoromethyl)phenyl)-3,6-dihydro-2H-1,4-oxazine. ¹H NMR (400 MHz, CDCl₃): 7.81 (d, J=8.29 Hz, 2H), 7.67 (d, J=8.29 Hz, 2H), 4.65 (t, J=2.49 Hz, 2H), 3.93 (m, 2H), 3.80 (m, 2H).

### (S)-Compound E

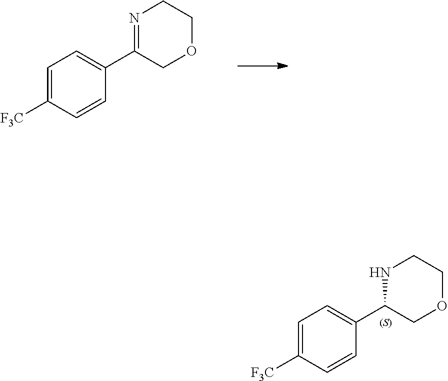

### Synthesis 1

Beta-nicotinamide adenine dinucleotide phosphate (NADP+) (753.4 mg, 1.013 mmol, 3 wt %), D-(+)-Glucose (26.1 g, 145 mmol, 1.4 equiv) and GDH-101 (754.8 mg, 3 wt %) were charged to a 100 mM potassium phosphate buffer, pH 7.4, (750 mL, 30 V) and stirred for approximately 10 to 15 minutes until all the solids were dissolved. IRED-155 (also identified as IRED-0712-C) (Prozomix) (2.533 g, 10 wt %) was charged and the reaction mass agitated for 10 to 15 minutes until all solids were in solution. The solution was heated to 30° C. and 5-(4-(trifluoromethyl)phenyl)-3,6-dihydro-2H-1,4-oxazine (23.5 g, 102.4 mmol, 1.0 equiv) was charged and the reaction was stirred for 18 hours. The reaction was cooled to 20° C. An aqueous solution of 6 N hydrochloric acid (61.0 mL, 2.6 V) was added over approximately 15 minutes until a pH of less than about 1.0 was obtained. The reaction mass was stirred for 2 hours. To the reaction mass was added filter agent (0.75 equiv by mass) and the mixture was agitated for one hour. The mixture was filtered over a pad of filter aid (0.25 equiv by mass) and washed with water. The filtrate was charged into the reactor and 10 N aqueous sodium hydroxide (51.6 mL, 2.2 equiv) was added over ≥15 minutes until a pH of 11 was obtained. After stirring for approximately 30 minutes, the mixture was filtered and dried under vacuum with nitrogen purge to afford (S)-3-(4-(trifluoromethyl)phenyl)morpholine ((S)-Compound E). (Analytical data: (+99% ee by chiral HPLC, LCMS: 232.08 (M+H). ¹H NMR (400 MHz, DMSO-d6): 7.68 (d, J=8 Hz, 2H), 7.65 (d, J=8 Hz, 2H), 3.89 (dd, J=9.95, 2.9 Hz, 1H), 3.74 (m, 2H), 3.47 (m, 1H), 3.15 (t, J=10.4 Hz, 1H), 2.95 (br s, 1H) 2.88 (m, 2H).

### Synthesis 2

To a reactor, 0.1 M potassium phosphate buffer (pH 6.4, 1.62 L, 13.5 L/kg) and D-glucose (132 g, 1.4 equiv) were charged. Nicotinamide adenine dinucleotide phosphate, mono-sodium salt (NADP, 1.8 g, 1.5 wt %), glucose dehydrogenase (GDH, 1.8 g, 1.5 wt %) and imine reductase (IRED, 6 g, 5 wt %) were all sequentially charged, then 5-(4-(trifluoromethyl)phenyl)-3,6-dihydro-2H-1,4-oxazine (120 g) was charged as a solid. Suitable IREDs include, for example, IRED-155 (also identified as IRED-0712-C) (Prozomix). Suitable GDHs include, for example, GDH-101. The disodium salt of NADP is also suitable. The reactor was heated to 30° C. The pH was continuously monitored, with potassium hydroxide (2 M) used to maintain the pH. A slow feed of NADP was added over the course of the reaction (1.8 g (1.5 wt %) NADP in 60 mL (0.5 L/kg) buffer). After 24 hours, the reaction mixture was diluted with acetonitrile (1.14 L, 9.5 L/kg) and aged with agitation for 10 minutes. 2-Methyltetrahydrofuran (900 mL, 7.5 L/kg) was then charged, then phases separated, and the aqueous layer was drained. The organic layer was washed with 20% w/w aqueous sodium chloride (600 mL, 5 L/kg), then distilled under vacuum to 360 mL. Isopropyl alcohol (1.44 L, 12 L/kg) was added and the mixture was distilled to 360 mL. Isopropyl alcohol (1.20 L, 10 L/kg) was added and the solution was polish filtered. Distill under vacuum to 480 mL Separately, acetyl chloride (45 mL, 1.2 equiv) was added dropwise to isopropanol (240 mL, 2 L/kg) at 0° C., then mixture heated to 20° C. and then aged for 15 minutes. Alternatively, HCL in a solvent (e.g., isopropanol, ethanol, or 2-methyltetrahydrofuran) could be added. The product/isopropanol mixture was heated to 60° C., and the HCl/isopropanol solution was charged slowly. The slurry was cooled to 20° C. Heptane (1.44 L, 12 L/kg) was charged over 2 h. Other suitable antisolvents include methyl ethyl ketone. The solid product was filtered and washed twice with pre-mixed 2:1 heptane:isopropanol (2×480 mL, 2×4 L/kg). The cake was vacuum dried under a stream of nitrogen to produce (S)-3-(4-(trifluoromethyl)phenyl)morpholine hydrochloride (>99.8% chiral purity). ¹H NMR (400 MHz, DMSO-d₆): Δ 9.80-10.99 (m, 2H), 7.95 (d, 2H), 7.83 (d, 2H), 4.61 (m, 1H), 4.02 (m, 2H), 3.86 (m, 2H), 3.24-3.32 (m, 2H); mp 198° C.

### Example 4. Synthesis of Compound I—(4-amino-1,3-dihydrofuro[3,4-c][1,7]naphthyridin-8-yl)-[(3S)-3-[4-(trifluoromethyl)phenyl]morpholin-4-yl]methanone

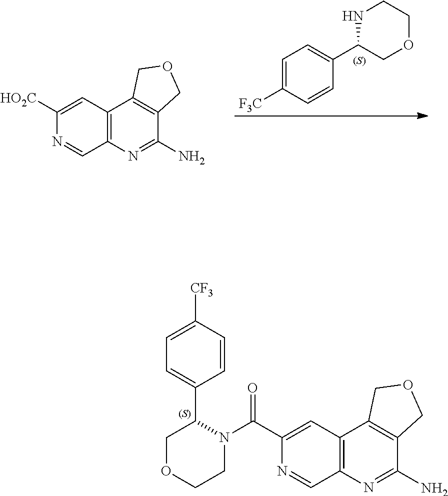

### Reaction Scale 1

4-Amino-1,3-dihydrofuro[3,4-c][1,7]naphthyridine-8-carboxylic acid (1.0 kg, 4.3 mol, 1.0 equiv), (3S)-3-[4-(trifluoromethyl)phenyl]morpholine (1.2 kg, 5.2 mmol, 1.2-equiv), and DMF, (6.6 kg, 7.0 V) were charged to a clean, dry reactor. To the mixture was added triethylamine (1.1 Kg, 13.8 mol, 2.6 equiv). The mixture was cooled to 10±5° C. and O-(benzotriazol-1-yl)-N,N,N′,N′-tetramethyluronium tetrafluoroborate (TBTU) (1.67 kg, 5.2 mol, 1.2 equiv) was added slowly. Next, an additional amount of DMF (0.94 Kg, 1 V) was added. The reaction mixture was warmed to 25±5° C. and stirred over 18 hours. Water (1.0 kg, 1 V) was charged followed by MeCN (1.6 kg, 2 V) and the reaction mass was warmed to 45° C. Next, water (7.0 Kg, 7 V) was added over 30 min. A seed lot of 4-amino-1,3-dihydrofuro[3,4-c][1,7]naphthyridin-8-yl)-[(3S)-3-[4-(trifluoromethyl)phenyl]morpholin-4-yl]methanone (10 g, 22 mmol, 0.01 equiv), was charged and the mixture was stirred at 45° C. for over 2 hours before being cooled to 20° C. over 10 hours. Water (12.0 kg, 12 V) was added over 2 hours at 20° C. and further stirred for over 4 hours before being filtered. The reactor was rinsed with a mixture of 10% DMF in water (9.83 kg, 10 V) and the resulting rinse mixture was used to wash the cake. The reactor was rinsed with a mixture of water (10.0 k kg, 10 V) and the resulting rinse mixture was used to wash the cake. This rinsing and washing protocol was repeated once more with water (10.0 k kg, 10V). The cake was dried under vacuum with a stream of nitrogen to afford (4-amino-1,3-dihydrofuro[3,4-c][1,7]naphthyridin-8-yl)-[(3S)-3-[4-(trifluoromethyl)phenyl]morpholin-4-yl]methanone. LCMS: 445.20 ¹H NMR (400 MHz, DMSO-d6 at 130° C.): 8.87 (s, 1H), 7.80 (s, 1H), 7.73 (d, J=8.7 Hz, 2H), 7.71 (d, J=8.7 Hz, 2H), 6.58 (br s, 2H), 5.72 (br s, 1H), 5.38 (m, 2H), 5.09 (t, J=3.5 Hz, 2H), 4.44 (br d, J=12.3 Hz, 1H), 4.08 (br d, J=13.4 Hz, 1H), 3.96 (dd, J=12.3, 3.7 Hz, 1H), 3.86 (br dd, J=11.4, 3.0 Hz, 1H), 3.66 (td, J=11.4, 3.0 Hz, 1H), 3.28 (m, 1H).

### Reaction Scale 2

4-Amino-1,3-dihydrofuro[3,4-c][1,7]naphthyridine-8-carboxylic acid (85.0 g, 352.2 mmol, 1.0 equiv), (3S)-3-[4-(trifluoromethyl)phenyl]morpholine (99.6 g, 422.6 mmol, 1.2-equiv), and DMF, (674 mL, 8.7 mol, 7.9 V) were charged to a clean, dry 5 L reactor. To the mixture was added 1-methylimidazole (75.2 g, 916.2 mmol, 2.6 equiv). The mixture was cooled to 0° C. and N,N,N′,N′-tetramethylchloroformamidinium hexafluorophosphate (TCFH) (118.6 g, 422.6 mmol, 1.2 equiv) was added slowly. Next, an additional amount of DMF (170 mL, 2 V) was added at 0° C. The reaction mixture was warmed to 25° C. and stirred overnight. Next, the reaction mass was warmed to 45° C. and 2-methyltetrahydrofuran, (169.2 mL, 2 V) was added followed by slow addition of water (850 mL, 10 V) over 30 min by addition funnel. A seed lot of 4-amino-1,3-dihydrofuro[3,4-c][1,7]naphthyridin-8-yl)-[(3S)-3-[4-(trifluoromethyl)phenyl]morpholin-4-yl]methanone (1.6 g, 3.5 mmol, 0.1 equiv), was charged as a slurry in a 1:1 v/v of DMF and water (31.3 mL) and the mixture was stirred at 45° C. for approximately 12 hrs. Water (510 mL, 6 V) was added over 1 h 10 min by addition funnel and the mixture was further stirred at 45° C. for 30 min before being filtered. The reactor was rinsed with water (340 mL, 4 V) and the resulting rinse mixture was used to wash the cake. This rinsing and washing protocol was repeated twice more. The cake was dried under vacuum with a stream of nitrogen to afford (4-amino-1,3-dihydrofuro[3,4-c][1,7]naphthyridin-8-yl)-[(3S)-3-[4-(trifluoromethyl)phenyl]morpholin-4-yl]methanone. LCMS: 445.20 ¹H NMR (400 MHz, DMSO-d6 at 130° C.): 8.87 (s, 1H), 7.80 (s, 1H), 7.73 (d, J=8.7 Hz, 2H), 7.71 (d, J=8.7 Hz, 2H), 6.58 (br s, 2H), 5.72 (br s, 1H), 5.38 (m, 2H), 5.09 (t, J=3.5 Hz, 2H), 4.44 (br d, J=12.3 Hz, 1H), 4.08 (br d, J=13.4 Hz, 1H), 3.96 (dd, J=12.3, 3.7 Hz, 1H), 3.86 (br dd, J=11.4, 3.0 Hz, 1H), 3.66 (td, J=11.4, 3.0 Hz, 1H), 3.28 (m, 1H).

### Reaction Scale 3:

4-Amino-1,3-dihydrofuro[3,4-c][1,7]naphthyridine-8-carboxylic acid (Compound A′) (20.0 g, 86.5 mmol, 1.0 equiv) was added to dimethylsulfoxide (400 mL) at 20° C. To the mixture was added 1,1′-carbonyldiimidazole (15.4 g, 95.2 mmol, 1.1 equiv) and the mixture was heated to 60° C. for 1 hour. A solution of (S)-3-(4-(trifluoromethyl)phenyl)morpholin-4-ium chloride (25.5 g, 95.2 mmol, 1.1 equiv) and dimethylsulfoxide (40 mL) was added, and the mixture was heated to 80° C. for 11 hours. The reaction mixture was cooled to 35° C., then water (265 mL) was added, then the batch was cooled to 20° C. The reaction was filtered, washed with 40% water:DMSO (80 mL), then washed with water (100 mL). The cake was dried under vacuum with a stream of nitrogen to afford (4-amino-1,3-dihydrofuro[3,4-c][1,7]naphthyridin-8-yl)-[(3S)-3-[4-(trifluoromethyl)phenyl]morpholin-4-yl]methanone (Compound I). LCMS: 445.20 ¹H NMR (400 MHz, DMSO-d6 at 130° C.): 8.87 (s, 1H), 7.80 (s, 1H), 7.73 (d, J=8.7 Hz, 2H), 7.71 (d, J=8.7 Hz, 2H), 6.58 (br s, 2H), 5.72 (br s, 1H), 5.38 (m, 2H), 5.09 (t, J=3.5 Hz, 2H), 4.44 (br d, J=12.3 Hz, 1H), 4.08 (br d, J=13.4 Hz, 1H), 3.96 (dd, J=12.3, 3.7 Hz, 1H), 3.86 (br dd, J=11.4, 3.0 Hz, 1H), 3.66 (td, J=11.4, 3.0 Hz, 1H), 3.28 (m, 1H).

### Recrystallization of Compound I

A clean, dry 5 L reactor was charged with (4-amino-1,3-dihydrofuro[3,4-c][1,7]naphthyridin-8-yl)-[(3S)-3-[4-(trifluoromethyl)phenyl]morpholin-4-yl]methanone (279.7 g, 0.6 mol, 1.0 equiv) followed by acetone (6.2 L, 22 V). The mixture was stirred at 40° C. for 15 minutes before cooling to 25° C. The reactor was discharged into a flask and the reactor was rinsed with acetone and the process stream was polish-filtered back into the reactor. The reactor jacket was set to 65° C. and the reaction volume was reduced to approximately 6 V by distillation at atmospheric pressure, crystallization was observed. The reaction temperature was set to cool to 20° C. over two hours. Heptane (2.8 L, 10 V) was added over two hours. The slurry was filtered and the cake was washed twice with a 4:1 Heptane/acetone mix (750 mL, 3 V each) and dried under vacuum with a nitrogen purge to afford (4-amino-1,3-dihydrofuro[3,4-c][1,7]naphthyridin-8-yl)-[(3S)-3-[4-(trifluoromethyl)phenyl]morpholin-4-yl]methanone.

### Example 5. Synthesis of Compound B1

This example demonstrates processes for preparing Compound B1 according to embodiments of the disclosure.

2-Cyano-5-nitropyridine was admixed with nitroreductase NR-17, glucose dehydrogenase GDH-101, NH₄VO₃ as a third transition metal catalyst, NADP as a co-factor, glucose as a reductant, and a buffer under reaction conditions A or B as set forth below and Table 2:

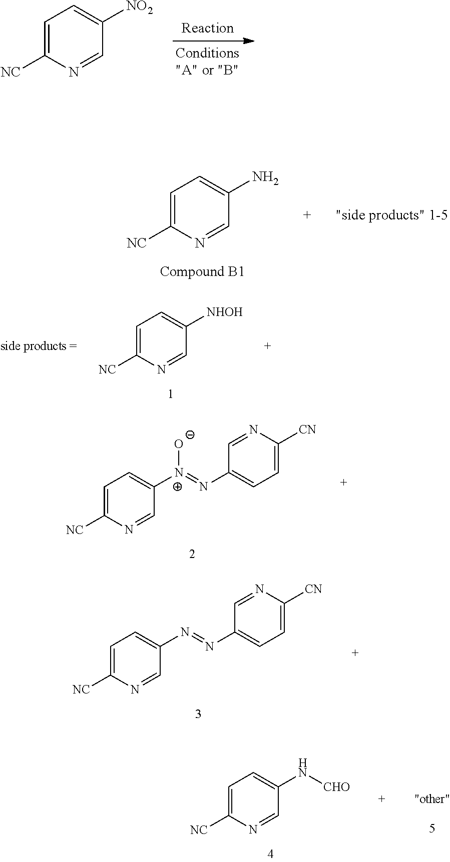

TABLE 2

| Reaction | Reaction | Yield | Side Product Yield (%) | | | | |
|---|---|---|---|---|---|---|---|
| Conditions | time (h) | B1 (%) | 1 | 2 | 3 | 4 | 5 |
| A | 2 | 81 | nd | 15 | <1 | 2 | 2 |
| B | 18 | 95 | nd | nd | nd | 5 | nd |
| B | 24 | 88 | <1 | nd | <1 | 10 | 2 |
| B | 41 | 86 | <1 | nd | <1 | 10 | 2 |
| B | 49 | 88 | <1 | <1 | 1 | 9 | 2 |
| B | 56 | 80 | <1 | <1 | <1 | 15 | 3 |

Reaction conditions A were as follows: 10 mg 2-cyano-5-nitropyridine; NR-17 (1 wt %); NH₄VO₃ (1 eq); NADP+(14 wt %); GDH (19 wt %); glucose (4 eq); DMSO (19 V); tricine buffer (170 V; pH 8); 35° C.; and reaction time of 2 h.

Reaction conditions B (fed-batch) were as follows: 2 g 2-cyano-5-nitropyridine in 0.7 V DMSO added over 63 h; NR-17 (7 wt %); NH₄VO₃ (16 mol %); NADP+(1 wt %); GDH (1 wt %); glucose (3 eq); KPi buffer (9 V; pH 7.2); 35° C., and reaction time of 18-56 h.

### Example 6. Synthesis of Compound B1

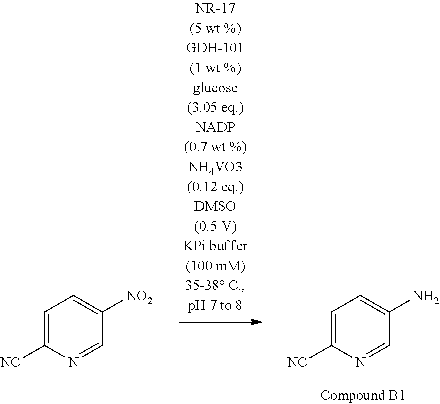 [SMILES: N#Cc1ccc([N+](=O)[O-])cn1 ; N#Cc1ccc(N)cn1]

This example demonstrates the synthesis of Compound B1 according to embodiments of the disclosed processes.

To a slurry of nitroreductase (NR-17; 250 mg, 5 mg/mL %), glucose dehydrogenase (GDH-105; 50 mg, 1 mg/mL %), co-factor (NADP; 36 mg, 1 mM), a third transition metal catalyst (NH₄VO₃; 468 mg, 0.12 eq) in buffer (KPi Buffer; 30 mL, 100 mM) at a pH of approximately 7.5 and a temperature of 20-25° C. was added a reductant (D-Glucose; 18.2 g, 3.05 eq) at a temperature of 20-25° C. The reaction mixture was admixed for 10-15 min. The pH of the reaction mixture was maintained at approximately 7.5 using base (e.g., 40% NaOH solution). The reaction mixture was heated to a temperature of 35 to 38° C. (internal temperature). To this heated mixture was added a solution of 2-cyano-5-nitropyridine (5 g, 33.5 mmol, 100 mass %) in DMSO (2.5 mL, 0.5 V) (total solution volume 5.5 mL) over a period of 6 h (e.g., using syringe pump at a flow rate of 0.015 mL/min) while maintaining the pH of the mixture at 7-8 using a base (e.g., 40% NaOH solution).

The reaction mass was stirred for 16 h at a temperature of 35-38° C. The progress of the reaction was monitored by HPLC. IPC by HPLC: Starting material=4.1% and Product=86%. Reaction was cooled to a temperature of 20-25° C. and quenched with water (30 V), and stirred for 10-15 min. The pH of the reaction mixture was adjusted to about 10. The reaction mixture was filtered to remove undissolved particles (solid wt: 0.3 g). The aqueous filtrate was extracted with organic solvent (e.g., (3×10 V of 2-methyltetrahydrofuran). The combined organic layers were washed Combined all organic layers and washed with water (10 V), dried over sodium sulfate, and concentrated under vacuum at 40-45° C. to provide 2.5 g Compound B1 (free base).

### Example 7—Synthesis of Compound B2

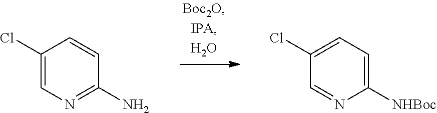 [SMILES: Nc1ccc(Cl)cn1 ; CC(C)(C)OC(=O)Nc1ccc(Cl)cn1]

To a reactor was charged solid di-tert-butyl carbonate (8.1 g, 1.2 equiv) and solid 6-chloropyridine-3-amine (4.0 g, 31 mmol). After purging with nitrogen, isopropanol (20 mL, 5.0 L/kg) was added and the mixture was heated to 55-60° C. Other suitable solvents include tert-butanol or tert-amyl alcohol. After 19 h, the reaction mixture diluted with water (35 mL, 8.75 L/kg) and the mixture was held at 60° C. for 1 hour. The reaction mixture was slowly cooled to 20° C., aged for 2 hours, filtered, then displacement-washed with 35% i-PrOH/water (24 mL, 6 L/kg). The solid was dried at ambient temperature under nitrogen sweep to yield tert-butyl (6-chloropyridine-3-yl)carbamate. ¹H NMR (400 MHz, CDCl₃): Δ 8.28 (d, 1H), 7.97 (bs, 1H), 7.28 (d, 1H), 6.79 (bs, 1H), 1.53 (s, 9H); mp 128° C.

### Example 8—Synthesis of Compound C′-6 tert-butyl (6-chloro-4-(1,3,6,2-dioxazaborocan-2-yl)pyridin-3-yl)carbamate

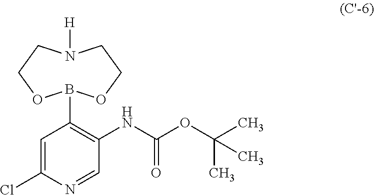 [SMILES: CC(C)(C)OC(=O)Nc1cnc(Cl)cc1B1OCCNCCO1]

A mixture of tert-butyl (6-chloropyridin-3-yl)carbamate (3.0 g, 13.1 mmol, 1.0 equiv), methylmagnesium chloride (3.0 M in tetrahydrofuran, 47.2 mmol, 3.6 equiv), 2,2,6,6-tetramethylpiperidine (6.66 g, 47.2 mmol, 3.6 equiv), lithium chloride (665 mg, 15.7 mmol, 1.2 equiv), tetrahydrofuran (7 mL) and 1,2-dimethoxyethane (38 mL) was stirred at 25° C. for >24 hours until reaction completion. A solution of triethylborate (7.27 g, 49.8 mmol, 3.8 equiv) in tetrahydrofuran (9 mL) was added. The reaction mixture was poured onto 60 mL of aqueous potassium sodium tartrate and 2-methyltetrahydrofuran, and the layers were separated. The organic layer was washed with water, then distilled to remove 1,2-dimethoxyethane and tetrahydrofuran. To the product stream in 2-methyltetrahydrofuran (approx. 30 mL) was added a solution of diethanolamine (1.52 g, 1.44 mmol, 1.1 equiv) in isopropanol (15 mL). Heptane (30 mL) was added, and the reaction mixture was filtered. The product cake was washed with 50% 2-methyltetrahydrofuran/heptane (30 mL), and dried at ambient temperature under a nitrogen sweep to afford the product tert-butyl (6-chloro-4-(1,3,6,2-dioxazaborocan-2-yl)pyridin-3-yl)carbamate (Compound C′-6). ¹H NMR (400 MHz, DMSO-d6): 9.51 (s, 1H), 8.77 (s, 1H), 7.50 (bs, 1H), 7.25 (bs, 1H), 3.70-3.95 (m, 4H), 3.17-3.23 (m, 2H), 3.02-3.09 (m, 2H), 1.46 (s, 9H).

### Example 9—Synthesis of 8-Chloro-1,3-dihydrofuro[3,4-c][1,7]naphthyridin-4-amine (A2)

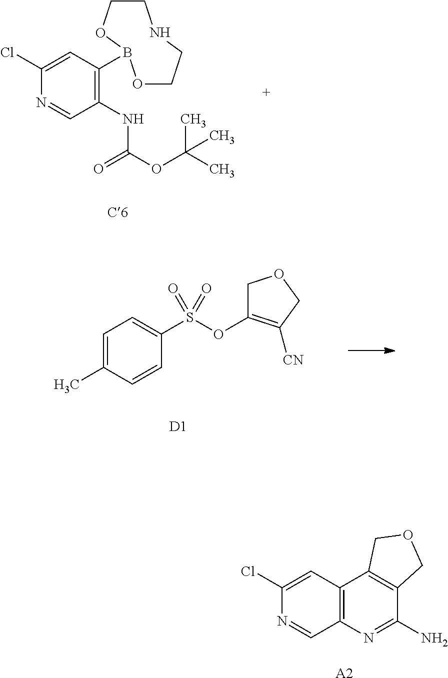

A sample of tert-butyl (6-chloro-4-(1,3,6,2-dioxazaborocan-2-yl)pyridin-3-yl)carbamate (Compound C′-6) (2.0 g, 5.85 mmol, 1.0 equiv) was combined with 2-methyltetrahydrofuran (36 mL), and 4-cyano-2,5-dihydrofuran-3-yl 4-methylbenzenesulfonate (Compound D) (2.33 g, 8.78 mmol, 1.5 equiv) was added. The mixture was washed twice with 2.5% aqueous acetic acid (18 mL). To the organic product layer were added bis(1,5-cyclooctadiene)nickel (0) (57 mg, 0.176 mmol, 3 mol %), tributylphosphonium tetrafluoroborate (153 mg, 0.527 mmol, 9 mol %), triethylamine (59 mg, 0.585 mmol, 0.1 equiv), water (10 mL) and potassium phosphate (2.5 g, 11.7 mmol, 2.0 equiv). The reaction was heated to 60° C. The batch was filtered, washed with water (10 mL), isopropanol (10 mL), and tert-butyl methyl ether (10 mL). The product cake was then dried under vacuum and nitrogen stream to afford the product 8-chloro-1,3-dihydrofuro[3,4-c][1,7]naphthyridin-4-amine (Compound A2)¹H NMR (400 MHz, Acetonitrile-d3): 8.69 (s, 1H), 7.47 (s, 1H), 6.52 (br s, 2H), 5.27 (br t, J=3.66 Hz, 2H), 5.03 (br t, J=3.66 Hz, 2H).

### Example 10—Synthesis of Compound A′—4-Amino-1,3-dihydrofuro[3,4-c][1,7]naphthyridine-8-carboxylic

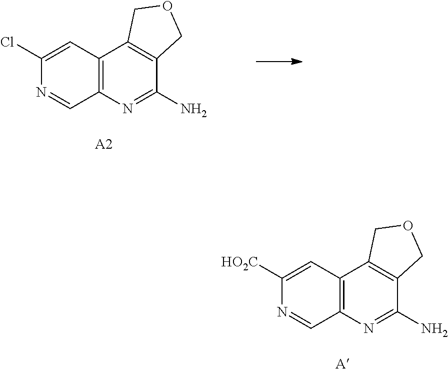

8-Chloro-1,3-dihydrofuro[3,4-c][1,7]naphthyridin-4-amine (Compound A2) (20.0 g, 90.2 mmol, 1.0 equiv), dimethylsulfoxide (900 mL), palladium(II) acetate (506 mg, 2.26 mmol, 2.5 mol %), 1,3-bis(dicyclohexylphosphino)propane bis(tetrafluoroborate) (1.38 g, 2.26 mmol, 2.5 mol %), water (24 mL), phenol (25.5 g, 271 mmol, 3.0 equiv) and potassium carbonate (62.3 g, 451 mmol, 5.0 equiv) were combined under 50 pounds-per-square-inch of carbon monoxide and heated to 85° C. for 21 hours. The mixture was cooled and nitrogen atmosphere was introduced. The batch was diluted with water (450 mL), filtered and washed with 33% water:DMSO (100 mL). To the resulting product-solid was added water (700 mL) at 48° C., then hydrochloric acid (6 N, 100 mL) was added. The batch was cooled to 20° C., filtered and washed with water (100 mL), isopropanol (100 mL) and tert-butyl methyl ether (100 mL). The product cake was then dried under vacuum and nitrogen stream to afford the product 4-Amino-1,3-dihydrofuro[3,4-c][1,7]naphthyridine-8-carboxylic acid (Compound A′). LCMS: 232.08 (M+H). ¹H NMR (400 MHz, 1:1 TFA-d/Toluene-d8): 9.30 (s, 1H), 8.29 (s, 1H), 5.11 (app s, 4H).

### Example 11—Compound A1—4-Amino-1,3-dihydrofuro[3,4-c][1,7]naphthyridine-8-carbonitrile

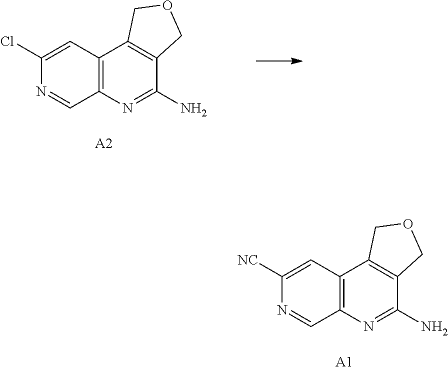

A mixture of bis(1,5-cyclooctadiene)nickel (0) (62 mg, 0.23 mmol, 5 mol %) and 4,5-bis(diphenylphosphino(-9,9-dimethylxanthene (Xantphos, 130 mg, 0.23 mmol, 5 mol %) in tetrahydrofuran (20 mL) was stirred for 20 min at 20° C. 8-Chloro-1,3-dihydrofuro[3,4-c][1,7]naphthyridin-4-amine (Compound A2) (1.0 g, 4.51 mmol, 1.0 equiv) and tetrahydrofuran (5 mL) were added and the solvents were stripped. The batch was diluted with dimethsulfoxide (30 mL) and 4-dimethylaminopyridine (550 mg, 4.51 mmol, 1.0 equiv) was added. To the batch were added zinc cyanide (425 mg, 3.61 mmol, 0.8 equiv) and zinc dust (88 mg, 1.35 mmol, 0.3 equiv). The reaction was heated to 80° C. for 16 hours. The reaction was cooled to ambient temperature, filtered and diluted with 2-methyltetrahydrofuran. The mixture was washed with aqueous ammonium hydroxide, then water, then the solvents were stripped. The residue was dissolved in N,N-dimethylacetamide (10 mL) and heptane (10 mL) was added. The slurry was filtered and washed with isopropanol (10 mL) to yield the product Compound A1—4-Amino-1,3-dihydrofuro[3,4-c][1,7]naphthyridine-8-carbonitrile. H NMR (400 MHz, TFA-d): 9.40 (s, 1H), 8.30 (s, 1H), 5.76 (d, J=3.2 Hz, 2H), 5.59 (s, 2H). LCMS: 213.1 (M+H)⁺.

All references, including publications, patent applications, and patents, cited herein are hereby incorporated by reference to the same extent as if each reference were individually and specifically indicated to be incorporated by reference and were set forth in its entirety herein.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

The use of the terms “a” and “an” and “the” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but notlimited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (for example, “such as”) provided herein, is intended merely to better illuminate the invention and does not pose a limitation on the scope of the invention unless otherwise claimed. No language in the specification should be construed as indicating any non-claimed element as essential to the practice of the invention.

1. A process for preparing Compound A, or a salt thereof: 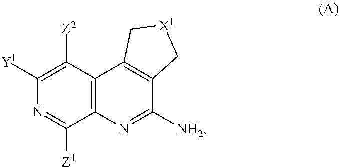
wherein
X¹ is NH, NR¹, O, S, or SO₂;
Y¹ is —CN, —Cl, —CHO, —COOH, —CONHR¹, —CON(R¹)₂, or —CO₂R¹;
each of Z¹ and Z² is independently H, F, or C₁-C₆ alkyl; and
each R¹ is independently C₁-C₆ alkyl;
comprising
(a) admixing Compound B with a first transition metal catalyst and a boron-containing compound to form Compound C when R^{B} is hydrogen or to form Compound C′ when R^{B} is —COOR₄, and optionally isolating Compound C or Compound C′: 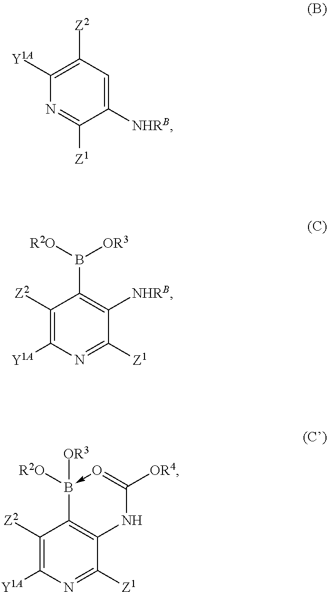
wherein R^{B} is hydrogen or —COOR⁴, each of R² and R³ is independently H or C₁-C₆ alkyl, or when taken together with the boron and oxygen atoms to which they are attached form a 5-, 6-, or 8-membered cyclic boronate; R⁴ is C₁-C₆alkyl; Y^{1A} is —CN, —Cl, —CONHR¹, —CON(R¹)₂, or —CO₂R¹; and
(b) admixing Compound C or Compound C′ with Compound D 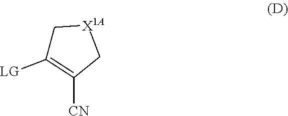
and a second transition metal catalyst to form Compound A or a salt thereof, wherein X^{1A} is NR⁷, O, or S, and R⁷ is C₁-C₆alkyl, benzyl, or p-methoxybenzyl; and LG is a leaving group.

2. The process of claim 1, wherein Compound A has a structure of A1 or A2:

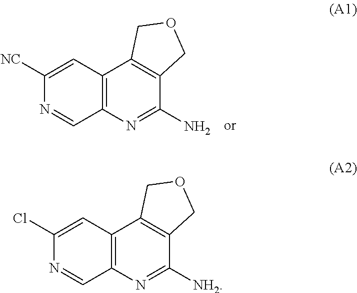 [SMILES: N#Cc1cc2c3c(c(N)nc2cn1)COC3 ; Nc1nc2cnc(Cl)cc2c2c1COC2]

3. The process of claim 1, wherein the first transition metal catalyst comprises iridium.

4. The process of claim 1, wherein the boron-containing compound is 4,4,4′,4′,5,5,5′,5′-octamethyl-2,2′-bi(1,3,2-dioxaborolane or 4,4,5,5-tetramethyl-1,3,2-dioxaborolane.

5. The process of claim 1, wherein Compound C′ has a structure of C′-4, C′-5, C′-6, or C′7:

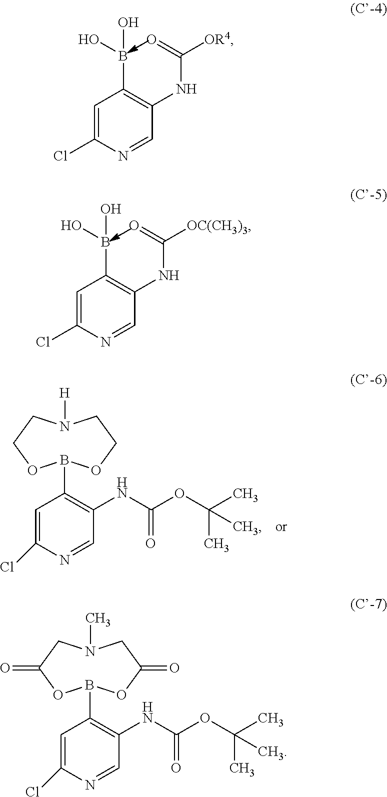

6. The process of claim 1, wherein Compound D has a structure of D1:

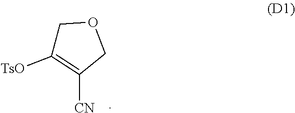 [SMILES: Cc1ccc(S(=O)(=O)OC2=C(C#N)COC2)cc1]

7. The process of claim 1, wherein the second transition metal catalyst is present in an amount of 1 to 5 mol % or wt %, based upon Compound B, and comprises a palladium catalyst or a nickel catalyst.

8. A process for preparing Compound E, a stereoisomer thereof, a salt thereof, or a salt of a stereoisomer thereof: 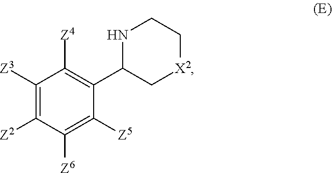
wherein
X² is NR¹, O, or S, R¹ is C₁-C₆alkyl;
Y² is H, C₁-C₆ alkyl, or C₁-C₆ haloalkyl; and
each of Z³, Z⁴, Z⁵, and Z⁶ is independently H, C1-C₆ alkyl, or chloride;
comprising
admixing Compound F, or a salt thereof, with an imine reductase (IRED) to form Compound E, a stereoisomer thereof, a salt thereof, or a salt of a stereoisomer thereof,

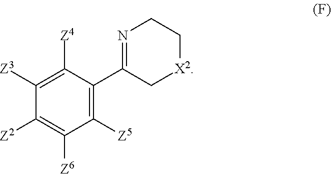

9. The process of claim 8, wherein X² is O, Y² is CF₃, and each of Z³, Z⁴, Z⁵, and Z⁶ is H.

10. The process of claim 8, wherein Compound E is enriched in the (S)-stereoisomer: 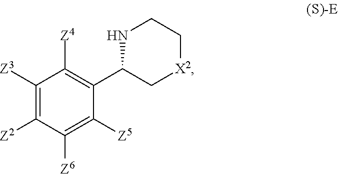
and Compound E has an enantiomeric excess of 95% or more.

11. The process of claim 8, further comprising
admixing Compound G or a salt thereof, with Compound H and an organometallic reagent or magnesium metal to form Compound F′, 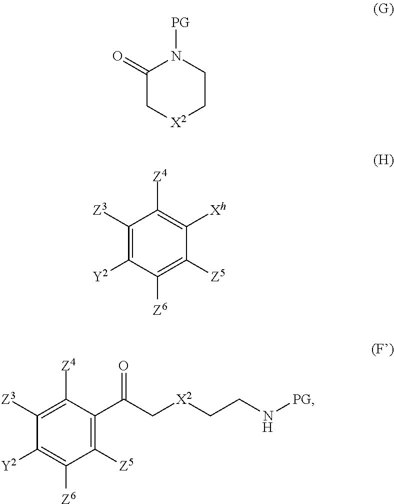
wherein PG is a protecting group and X^{h} is Cl, Br, or I.

12. The process of claim 11, wherein the organometallic reagent is iPrMgCl, and the protecting group is selected from the group consisting of tert-butyloxycarbonyl (Boc), benzyloxycarbonyl (Cbz), and trimethylsilyl (TMS).

13. The process of claim 11, further comprising deprotecting Compound F′ to form Compound F, or salt thereof.

14. A process for preparing Compound I, a stereoisomer thereof, a salt thereof, or a salt of a stereoisomer thereof: 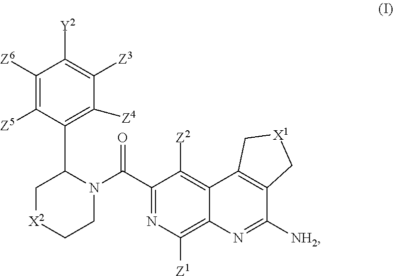
comprising
admixing Compound A′, or a salt thereof with Compound E, a stereoisomer thereof, a salt thereof, or a salt of a stereoisomer thereof, and a coupling agent to form Compound I, a stereoisomer thereof, a salt thereof, or a salt of a stereoisomer thereof, 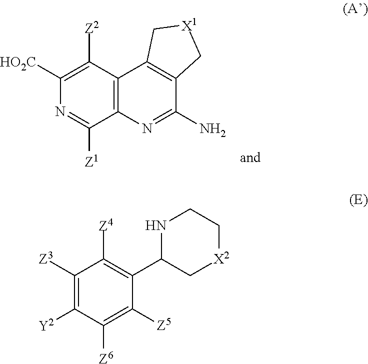
wherein
X¹ is NH, NR¹, O, S, or SO₂;
X² is NR¹, O, or S;
each R¹ is independently C₁-C₆alkyl;
Y² is H, C₁-C₆ alkyl, or C₁-C₆ haloalkyl;
each of Z¹ and Z² is independently H, F, or C₁-C₆alkyl; and
each of Z³, Z⁴, Z⁵, and Z⁶ is independently H, C₁-C₆alkyl, or chloride.

15. The process of claim 14, wherein X¹ and X² are each O; Y² is —CF₃; and each of Z¹, Z², Z³, Z⁴, Z⁵, and Z⁶ is H.

16. The process of claim 14, wherein the coupling agent is selected from the group consisting of chloro-N,N,N′,N′-tetramethylformamidinium hexafluorophosphate (TCFH), O-[(ethoxycarbonyl)cyanomethyleneamino]-N,N,N′N′-tetramethyluronium tetrafluoroborate (TOTU), 1-cyano-2-ethoxy-2-oxoethylidenaminooxy)dimethylamino-morpholino-carbenium hexafluorophosphate (COMU), 1-[bis(dimethylamino)methylene]-1H-1,2,3-triazolo[4,5-b]pyridinium 3-oxide hexafluorophosphate (HATU), N-[(1H-benzotriazol-1-yl)-(dimethylamino)methylene]-N methylmethanaminium hexafluorophosphate N-oxide (HBTU), O-(benzotriazol-1-yl)-N,N,N′,N′-tetramethyluronium tetrafluoroborate (TBTU), propanephosphonic acid anhydride (T3P), bis(2-oxo-3-oxazolidinyl)phosphinic chloride (BOPCl), 2-chloro-4,6-dimethoxy-1,3,5-triazine (CDMT), 1,1′-carbonyldiimidazole (CDI), and 1-cyano-2-ethoxy-2-oxoethylideneaminooxy-tris-pyrrolidino-phosphonium hexafluorophosphate (PyOxim).

17. The process of claim 16, wherein the coupling agent is O-(benzotriazol-1-yl)-N,N,N′,N′-tetramethyluronium tetrafluoroborate (TBTU).

18. The process of claim 16, wherein the coupling agent is CDI.

19. The process of claim 14, wherein the admixing is performed in the presence of an additive.

20. The process of claim 19, wherein the additive is N-methylimidazole (NMI) or triethylamine.

21. The process of claim 19, wherein the additive is trifluoromethanesulfonic acid, hydrochloric acid, hydrobromic acid, or hydroiodic acid.

22. The process of claim 14, further comprising crystallizing Compound I, a stereoisomer thereof, a salt thereof, or a salt of a stereoisomer thereof.

23. The process of claim 14, wherein Compound I has the structure:

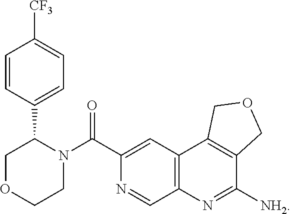 [SMILES: Nc1nc2cnc(C(=O)N3CCOC[C@@H]3c3ccc(C(F)(F)F)cc3)cc2c2c1COC2]

24. The process of claim 1, wherein Compound B has a structure of B1 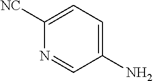
(Compound B1) or B1′ 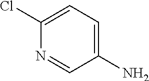
(Compound 1′).

25. The process of claim 24, further comprising admixing 2-cyano-5-nitropyridine 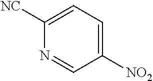 [SMILES: N#Cc1ccc([N+](=O)[O-])cn1]
or 2-chloro-5-nitropyridine 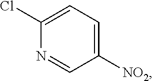
or a salt thereof with a nitroreductase in a solvent to form Compound 1, Compound 1′, or a salt thereof.

26. The process of claim 25, wherein the nitroreductase is NR-17 or NR-X36 and is present in an amount of 5-7 wt %, based upon 2-cyano-5-nitropyridine or 2-chloro-5-nitropyridine.

27. The process of claim 25, further comprising admixing 2-cyano-5-nitropyridine, 2-chloro-5-nitropyridine, or a salt thereof and the nitroreductase in the presence of one or more of a glucose dehydrogenase (GDH), a third transition metal catalyst, a co-factor, a reductant, or a buffer.

28. The process of claim 27, wherein the third transition metal catalyst comprises vanadium, iron, copper, or a combination thereof.

29. The process of claim 28, wherein the third transition metal catalyst is ammonium metavanadate (NH₄VO₃) or vanadium pentoxide (V₂O₅).

30. The process of claim 27, wherein the third transition metal catalyst is present in an amount of 0.01-2.5 eq, based upon 2-cyano-5-nitropyridine or 2-chloro-5-nitropyridine.

31. The process of claim 27, wherein the glucose dehydrogenase is selected from the group consisting of GDH-101, GDH-105, CDX-901, and a combination thereof.

32. The process of claim 27, wherein the reductant is glucose.

33. The process of claim 27, wherein the buffer comprises a tricine buffer, a potassium phosphate buffer, 4-(2-hydroxyethyl)-1-piperazineethanesulfonic acid (HEPES), tris(hydroxymethyl)aminomethane (Tris), or a combination thereof.

34. The process of claim 25, wherein the admixing of 2-cyano-5-nitropyridine, 2-chloro-5-nitropyridine, or salt thereof with the nitroreductase is conducted in a solvent comprising water, dimethylsulfoxide (DMSO), toluene, methyl tert-butyl ether (MTBE), isopropyl acetate, or a combination thereof.

35. The process of claim 34, wherein the solvent comprises 0.5-20 volumes of DMSO, based upon 2-cyano-5-nitropyridine.

36. The process of claim 25, wherein the admixing of 2-cyano-5-nitropyridine, 2-chloro-5-nitropyridine, or salt thereof with the nitroreductase is conducted at a temperature of 32-38° C.
